(19) United States
(12) Patent Application Publication
(10) Pub. No.: US 2024/0270704 A1
(43) Pub. Date: Aug. 15, 2024

(54) AMINO ACIDS BEARING A TETRAZINE MOIETY

(71) Applicant: VALANX Biotech GmbH, Klosterneuburg (AT)

(72) Inventor: Michael LUKESCH, Wien (AT)

(73) Assignee: VALANX Biotech GmbH, Klosterneuburg (AT)

(21) Appl. No.: 18/563,234

(22) PCT Filed: May 25, 2022
(86) PCT No.: PCT/EP2022/064273

§ 371 Date: Nov. 21, 2023

(30) Foreign Application Priority Data
May 26, 2021 (EP) 21175917.0

(51) Int. Cl. C07D 257/08; C07K 1/107

(52) U.S. Cl. CPC C07D 257/08; C07K 1/1075

(57) ABSTRACT

The invention relates to a novel amino acid having a tetrazine moiety and peptide or protein comprising the novel amino acid compounds. The invention also relates to a method of producing peptide or proteins comprising a tetrazine moiety and to the use of said peptide or proteins.

### FIELD OF THE INVENTION

The invention relates to novel amino acid derivatives bearing a tetrazine moiety, to a process for the preparation thereof and use of the novel compounds in click chemistry or site-specific protein or peptide modification.

### BACKGROUND ART

Peptides and proteins are a major product of the biotechnological industry. They are applied as therapeutics, detecting entities for diagnostics in vivo and in vitro as well as coatings on surfaces, e.g., for implants or biosensors. Peptides and proteins are used for these applications because they provide specific binding to therapeutic or diagnostic targets (Hu Q.-Y. et al. (2016)). In order to expand the capabilities of peptides and proteins, additional chemical entities like small molecules, polymers or other proteins/peptides are conjugated to them. Current conjugation approaches suffer from the drawback of being non-site specific. The process yields an undefined mixture of conjugates, where the location and the number of conjugation connections are randomly distributed and initially unknown. This poses a significant challenge in the manufacturing of these products since processes are random and unreliably and regulatory hurdles demanding precise reproducibility are harder to fulfil.

One of the most promising approaches to solve this challenge is the site-specific incorporation of synthetic, reactive amino acids into peptides and proteins. These synthetic amino acids carry chemical functional groups not found in natural peptides or proteins and are therefore orthogonal to their chemical reactions. By introducing a synthetic amino acid at a defined site into a peptide or protein and then reacting the synthetic amino acid with the desired additional chemical entity, site-specific conjugates that are precisely defined can be achieved.

For peptides, the site-specific incorporation is straight forward by placing the synthetic amino acid at the desired location by chemical synthesis of the peptide. Proteins on the other hand are manufactured in prokaryotic and eukaryotic biotechnological hosts. Achieving site-specific incorporation in these hosts requires the engineering of the central cellular process of protein synthesis. To this end, two orthogonal systems for synthetic amino acid incorporation have been developed, the tyrosyl-tRNA synthetase from Methanocaldococcus janaschii (Young T. S. et al. (2010)) and the pyrrolysyl-tRNA synthetase from Methanosarcina mazei/Methanosarcina Bakeri/Methanomethylophilus alvus (Wan W. et al. (2014)). While the tyrosyl-tRNA synthetase charges tyrosine to its cognate tRNA, the pyrrolysyl-tRNA synthetase charges pyrrolysine, an unusual amino acid specific to the genus Archaea.

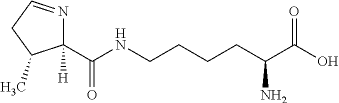 [SMILES: C[C@@H]1CC=N[C@H]1C(=O)NCCCC[C@H](N)C(=O)O]

### Structure of Pyrrolysine

These two systems are further subjugated to protein engineering approaches to evolve them for efficient incorporation of synthetic amino acids (Owens A. E. et al. (2017)). Since the tyrosyl-tRNA synthetase system incorporates tyrosine as its natural function, it is prone upon engineering efforts to continue to accept tyrosine as well as phenylalanine and other standard amino acids. Therefore, laborious negative selection screens must be applied to minimize this unwanted activity. However, in a lot of cases it remains as a background activity and leads to the production of different protein species with and without the synthetic amino acid incorporated. The pyrrolysyl-tRNA system does not suffer from this drawback since the structure of pyrrolysine is sufficiently different from all other standard amino acids to allow specific incorporation of only the lysine derivatives. A wide spectrum of synthetic amino acids has been incorporated using this system (Yanagisawa T. et al. (2018), Hohl A. et al. (2017)).

Synthetic amino acids being considered for incorporation into peptides and proteins need to fulfil a list of requirements to be deemed suitable for use in product development:

### Fast Reaction Speed

Proteins and peptides are delicate entities and need to be handled with care during the manufacturing process and kept at low temperatures and physiological conditions. Hence the conjugation reaction should also proceed irreversibly within these conditions, ideally without any catalyst added. The most suitable reaction known to date is the inverse-electron-demand Diels-Alder (iEDDA) reaction between tetrazines and strained dienophiles (Lang K. and Chin J. W. (2014)).

### Stability

Their reactiveness makes the used synthetic amino acids prone to degradation in solution. Ideally, the amino acids should be stable in biological and chemical incorporation conditions. Synthetic tetrazine amino acids should be of a certain structure to be stable biologically and chemically (Eising S. et al. (2018), Zeglis B. M. et al. (2014)).

### Ease and Efficiency of Incorporation

One drawback of the technology of synthetic amino acid incorporation is, that the protein production process is not as efficient as when producing a natural protein. The major determinant for protein yield is how well the engineered pyrrolysyl-system charges the synthetic amino acid to its tRNA. Certain structures provide a benefit in incorporation efficiency and therefore enable a higher yield production of the target protein leading to significant industrial competitive advantage.

### Solubility

For the synthetic amino acid to be used in an industrial fermentation process, its solubility in growth and fermentation media needs to be high. Additionally, it should be able to be dissolved in high concentration in a benign stock feed solution to feed it during the fermentation process. Solubility in growth and fermentation media as well as in benign stock feed solution is determined by chemical structure.

WO2014117001 discloses a modified amino acid bearing an unsubstituted tetrazine moiety. WO2014065860 discloses functionalized 1,2,4,5-tetrazine compounds. WO2016176689A1 discloses phenylalanine-derived tetrazine amino acids with only ˜50% efficiency of incorporation and low solubility. Mayer, S. V. et al. (2019) disclose lysine-derived tetrazine amino acids with low solubility and only ˜50% efficiency of incorporation.

Thus, there is still a need for novel synthetic amino acids bearing a tetrazine moiety which may react with various chemical groups. Specifically, there is the need for novel synthetic amino acids which react with very fast rates at physiological pH in aqueous conditions at room temperature, which show high solubility in growth and fermentation media as well as in benign stock solutions and exhibit high incorporation efficiencies of above 50%.

### SUMMARY OF INVENTION

It is an object of the present invention to provide novel synthetic amino acids bearing a tetrazine group. These novel synthetic amino acids exhibit high solubility in polar solvents and high incorporation efficiency in the production of proteins and polypeptides. The object is solved by the subject-matter of the present invention.

The present invention relates to novel synthetic amino acids bearing a tetrazine moiety, to methods for producing said synthetic amino acids and to their use.

One embodiment of the invention relates to compounds of general formula I,

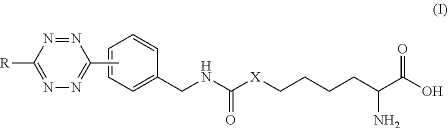

wherein
X denotes N or O,
R is selected from the group consisting of halogen, —OR^{a}, —C(O)R^{a}, —COOR^{a}, —NR^{a}R^{a}, —SR^{a}, —C₁₋₆alkyl and phenyl, wherein the —C₁₋₆alkyl or phenyl moiety is optionally substituted by halogen, —OR^{a}, —C(O)R^{a}, —COOR^{a}, —NR^{a}R^{a}, —SR^{a}, and R^{a} is hydrogen or C₁₋₆alkyl.

According to one embodiment of the invention, the compound of formula (I) is selected from the group consisting of:

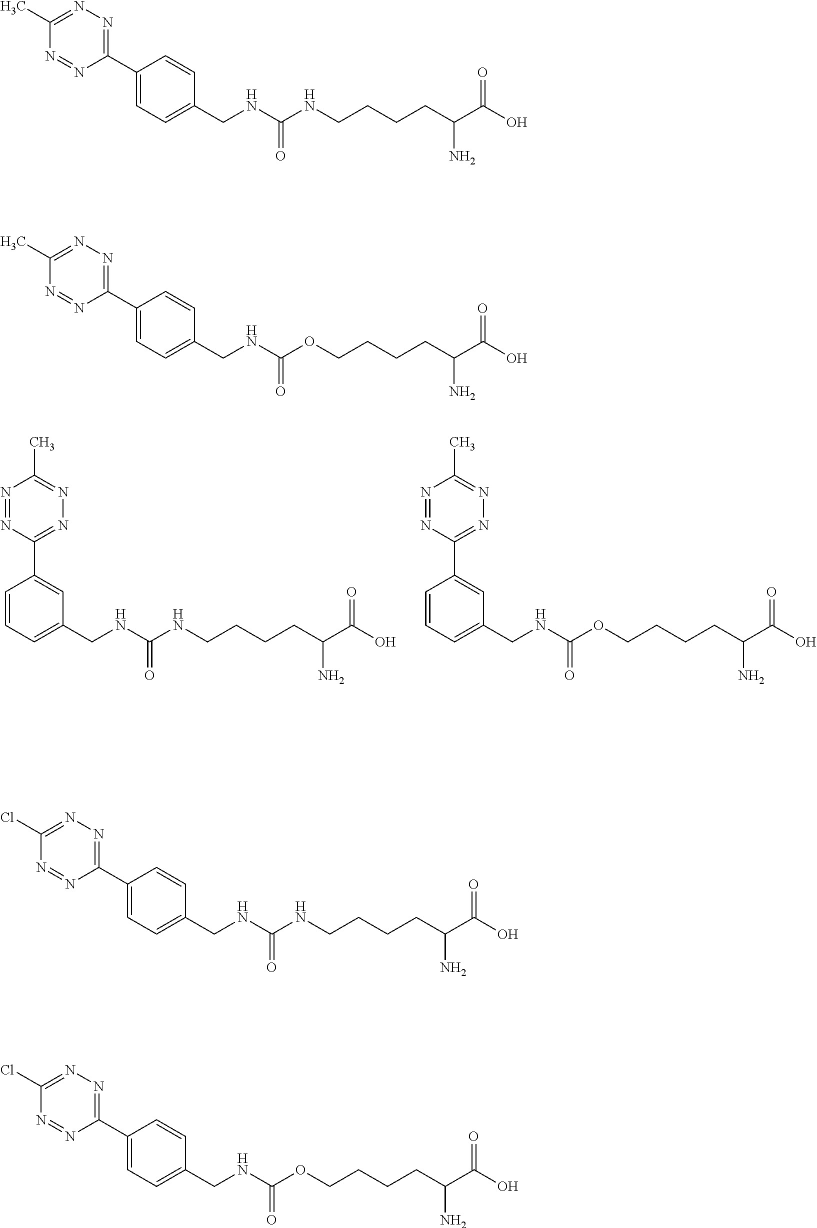 [SMILES: Cc1nnc(-c2ccc(CNC(=O)NCCCCC(N)C(=O)O)cc2)nn1 ; Cc1nnc(-c2ccc(CNC(=O)OCCCCC(N)C(=O)O)cc2)nn1 ; Cc1nnc(-c2cccc(CNC(=O)NCCCCC(N)C(=O)O)c2)nn1 ; Cc1nnc(-c2cccc(CNC(=O)OCCCCC(N)C(=O)O)c2)nn1 ; NC(CCCCNC(=O)NCc1ccc(-c2nnc(Cl)nn2)cc1)C(=O)O ; NC(CCCCOC(=O)NCc1ccc(-c2nnc(Cl)nn2)cc1)C(=O)O]

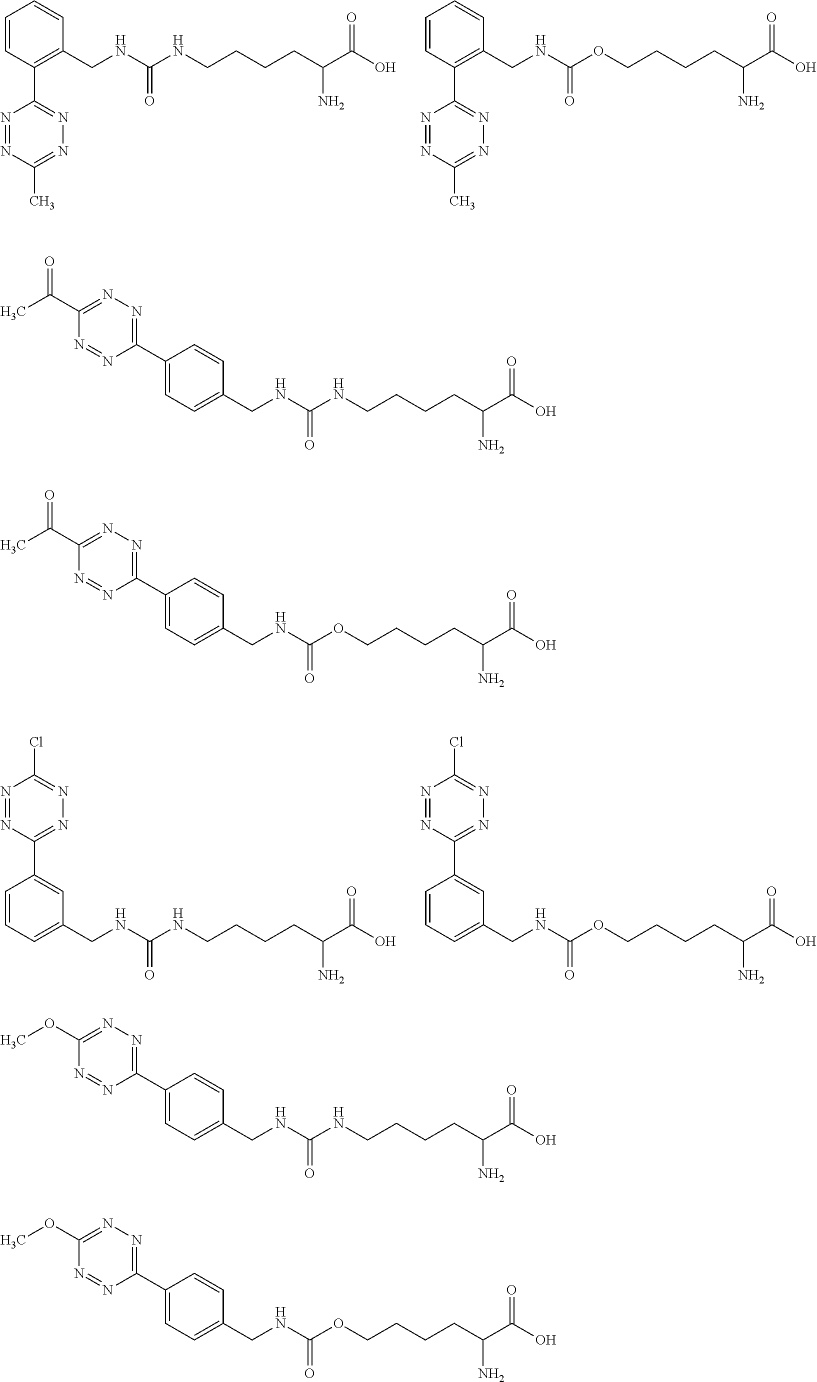

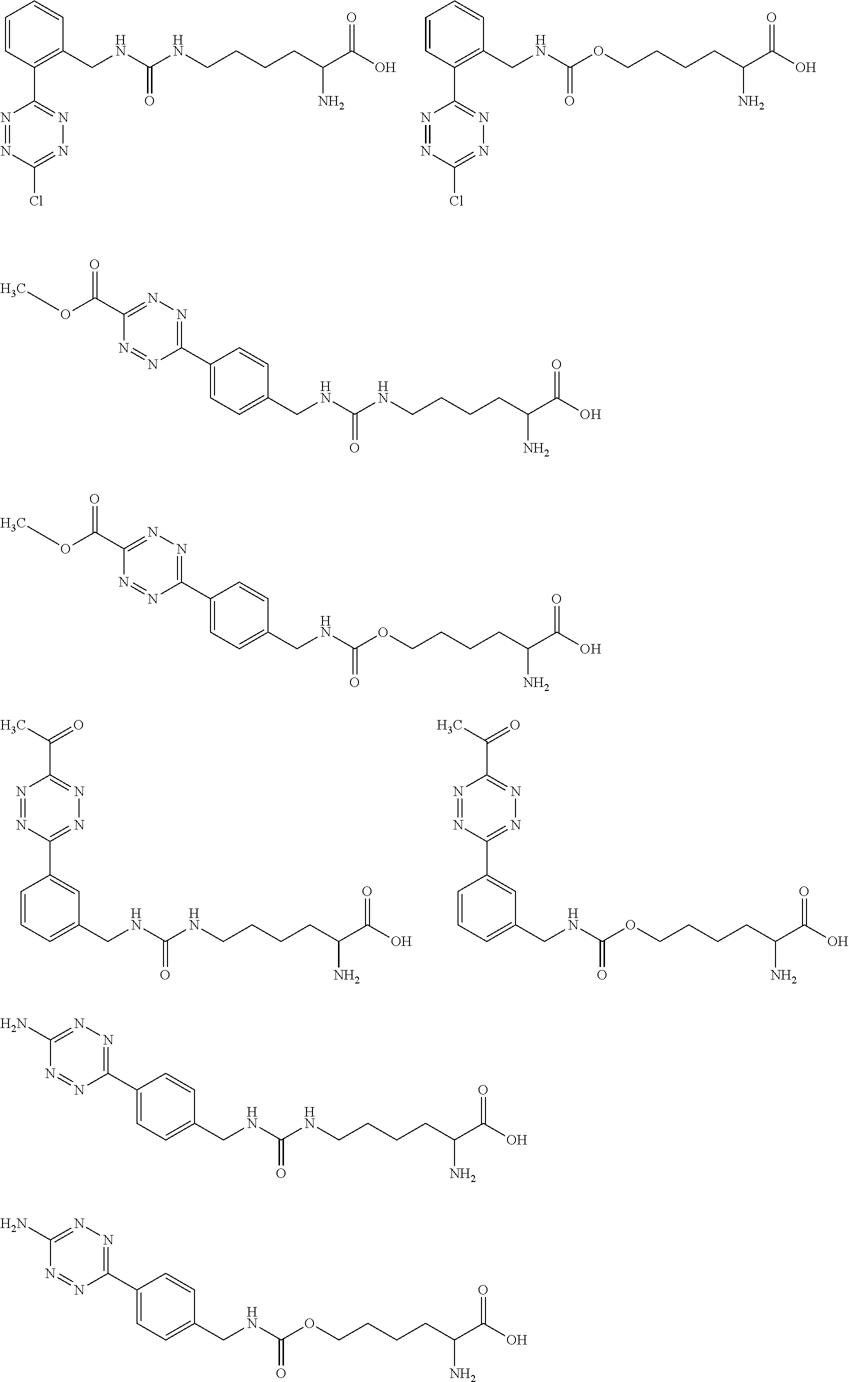

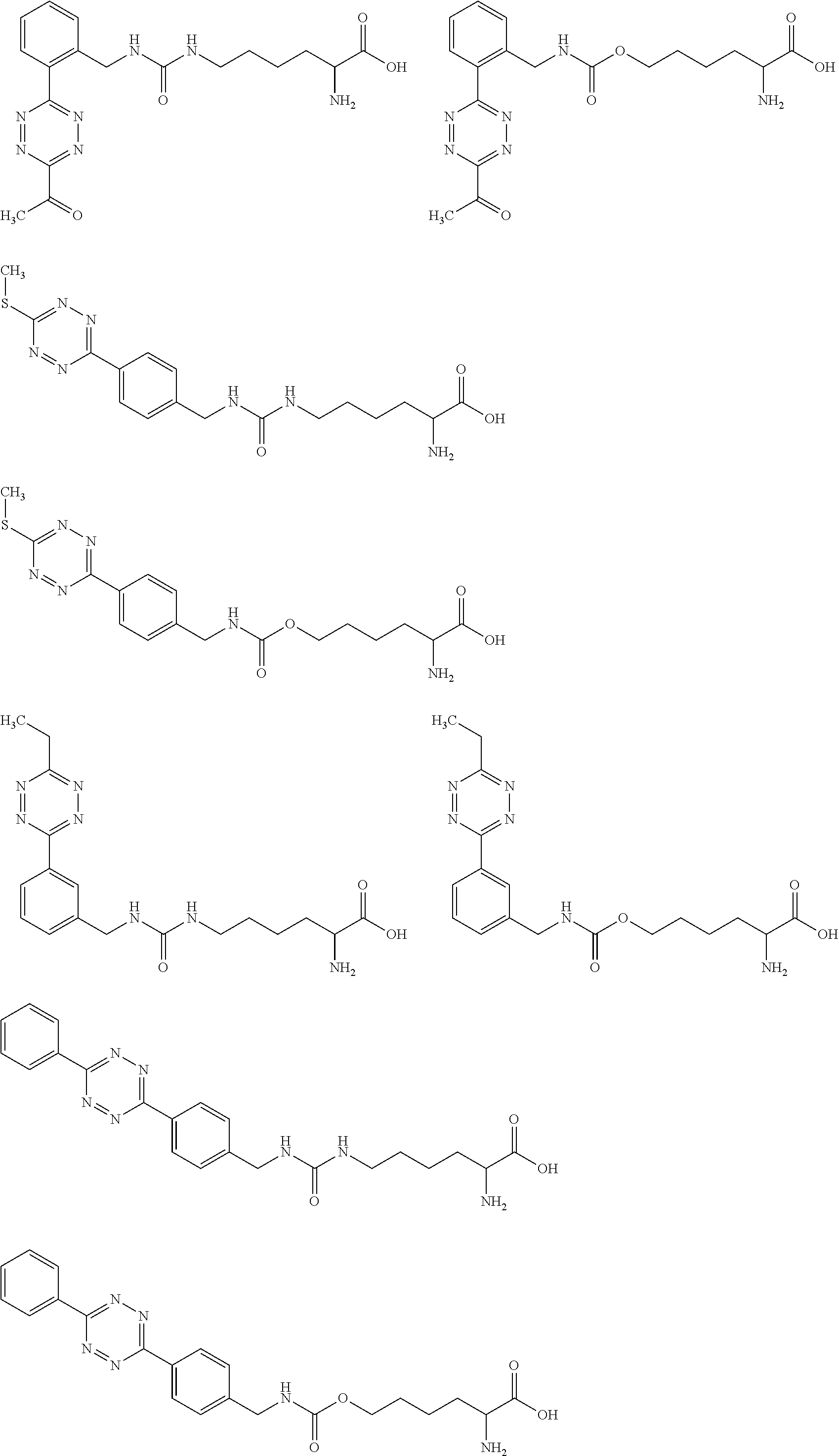

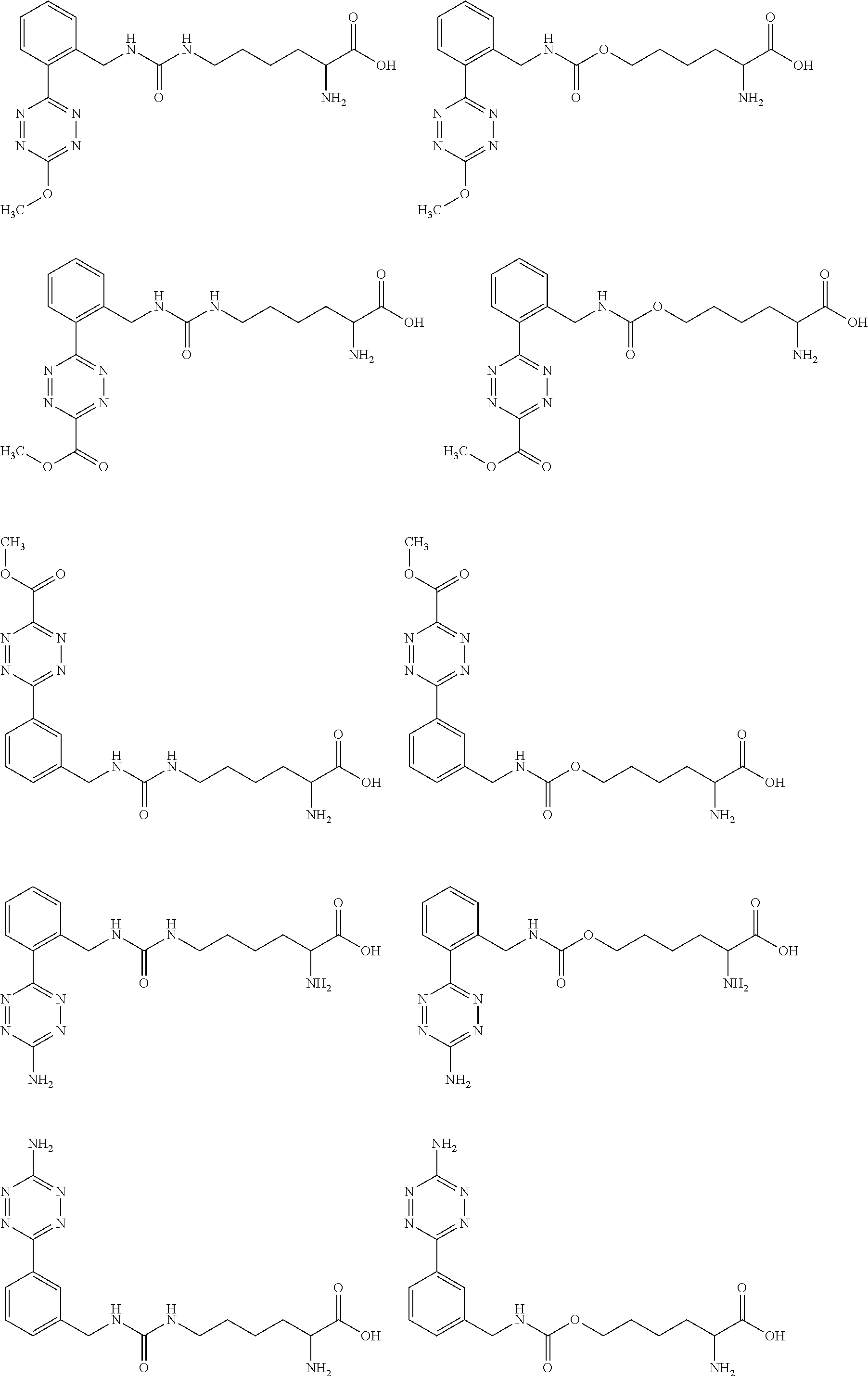

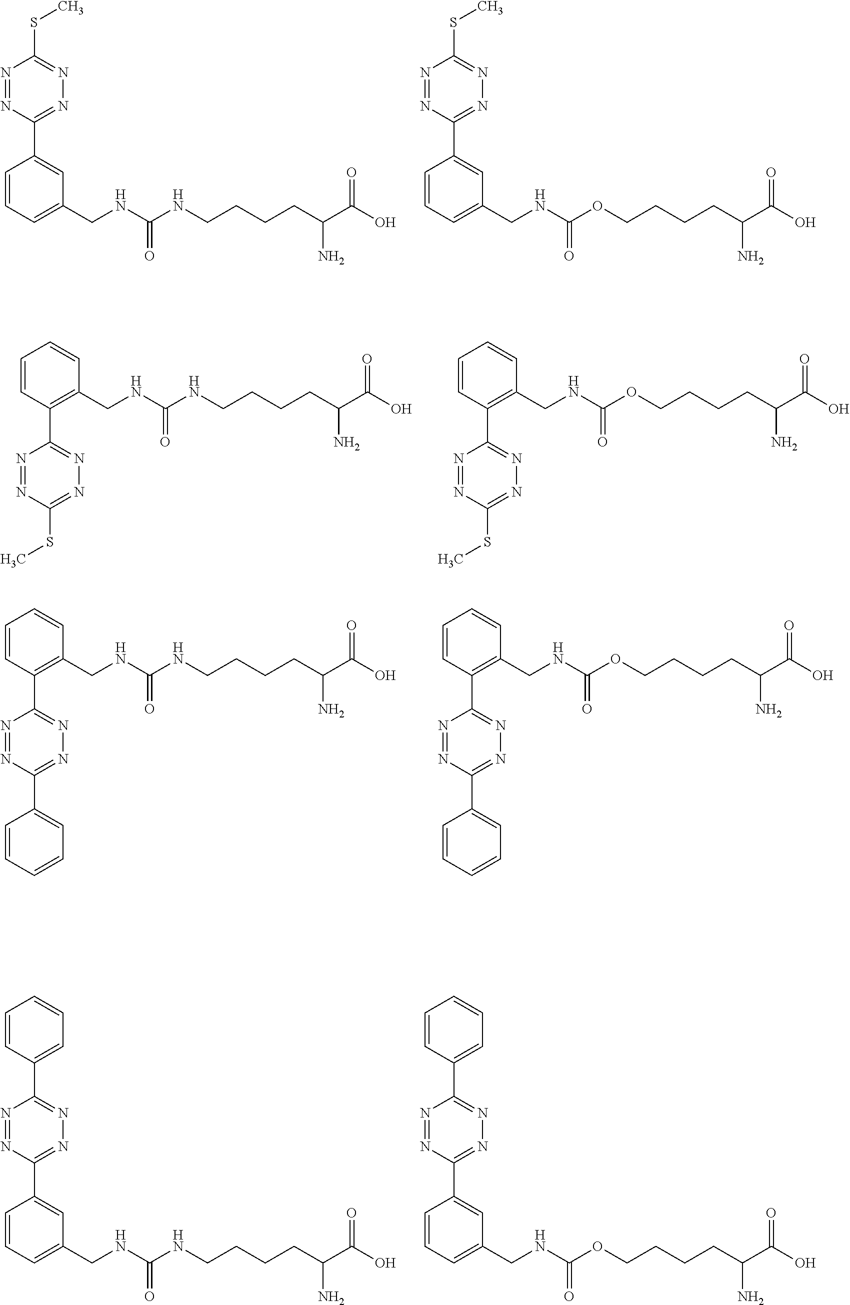

A further embodiment relates to the compound of formula (I), wherein R is methyl.

One embodiment of the invention relates to a method for producing compounds of general formula (I), comprising the step of reacting a tetrazine derivative of formula (II) with a lysine derivative of formula (III)

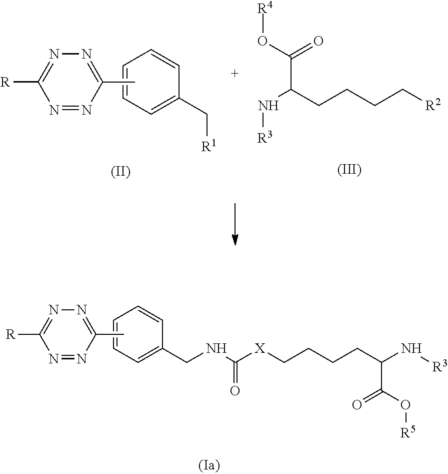

in order to achieve the intermediate compound (Ia), wherein
X denotes N or O,
R is selected from the group consisting of halogen, —OR^{a}, —C(O)R^{a}, —COOR^{a}, —NR^{a}R^{a}, —SR^{a}, —C₁-C₆alkyl and phenyl, wherein the —C₁-C₆alkyl or phenyl moiety is optionally substituted by halogen, —OR^{a}, —C(O)R^{a}, —COOR^{a}, —NR^{a}R^{a}, —SR^{a},
R¹ is —NH₂ or —OCN,
R² is —OH or —NH—C(═O)-imidazol,
R³ is a protecting group,
R⁴ is H or a protecting group.
R⁵ is —OH or —OCH₃, and
R^{a} is hydrogen or C₁₋₆alkyl

A further embodiment relates to the method as described herein, wherein the protecting group is selected from the group consisting of tert-butyloxycarbonyl (boc-group), carbobenzyloxy (Cbz) group, p-methoxybenzyl carbonyl (Moz or MeOZ) group, tert-butyloxycarbonyl (BOC) group, 9-fluorenylmethyloxycarbonyl (Fmoc) group, acetyl (Ac), benzoyl (Bz) group, benzyl (Bn) group, carbamate group, p-methoxybenzyl (PMB), 3,4-dimethoxybenzyl (DMPM), p-methoxyphenyl (PMP) group, tosyl (Ts) group, and Troc (trichloroethyl chloroformate).

One embodiment of the invention relates to a method for site-specific incorporation of a compound of formula (I) into a peptide or protein, comprising the steps of
providing a compound of formula (I),
providing a cell strain with an orthogonal tRNA-synthetase enzyme with tRNA-charging activity towards the compounds of formula (I),
culturing said cell strain in a culture medium comprising the compound of a), and
recovering from the culture medium or from the cells obtained in step c) the modified peptide or protein containing at least one compound of formula (I).

A further embodiment relates to a modified peptide or protein, wherein the peptide or protein comprises at least one compound of general formula (I).

According to a further embodiment, the modified peptide or protein as described herein comprises at least one compound of formula (I) which is incorporated at a desired position of the wild type peptide or protein.

Another embodiment of the invention relates to the modified peptide or protein as described herein, wherein the tetrazine moiety of said modified peptide or protein is further linked to an electron-rich dienophile compound or a strained dienophile compound.

One embodiment of the invention relates to the modified peptide or protein as described herein, wherein said electron-rich dienophile compound is selected from the group consisting of a norbornene compound, a cyclopropene compound, a bicyclo[6.1.0]nonyl compound, a trans-cyclooctene compound, a styrene compound or a spirohexene compound.

A further embodiment relates to the method of producing a modified peptide or protein, comprising the steps of:
providing a modified peptide or protein according to claim 7 or 8 and an electron-rich dienophile compound,
incubating said components to allow linkage of the tetrazine moiety of the modified peptide or protein to said electron-rich dienophile compound.

A further embodiment relates to the method as described herein, wherein said electron-rich dienophile compound is selected from the group comprising a norbornene compound, a cyclopropene compound, and a bicyclo[6.1.0]nonyl compound, a trans-cyclooctene compound, a styrene compound or a spirohexene compound.

One embodiment of the invention relates to the use of a compound of general formula (I) for chemical synthesis or as synthon for pharmaceutical ingredients.

A further embodiment of the invention relates to the use of a compound of general formula (I) as building block in chemistry or as synthon for pharmaceutical ingredients.

### DESCRIPTION OF EMBODIMENTS

The present invention relates to novel synthetic amino acids bearing a tetrazine moiety. This synthetic amino acid compounds are able to react with various groups, e.g., electron-rich dienophile or strained dienophile compounds, in click chemistry reactions at very fast rates at physiological pH in aqueous conditions at room temperatures.

Thus, one embodiment of the invention relates to novel amino acid compounds of general formula (I),

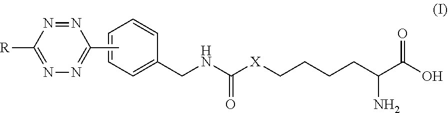

wherein
X denotes N or O, and
R is selected from the group consisting of halogen, —OR^{a}, —C(O)R^{a}, —COOR^{a}, —NR^{a}R^{a}, —SR^{a}, —C₁₋₆alkyl and phenyl, wherein the —C₁₋₆alkyl or phenyl moiety is optionally substituted by halogen, —OR^{a}, —C(O)R^{a}, —COOR^{a}, —NR^{a}R^{a}, —SR^{a}, and R^{a} is hydrogen or C₁₋₆alkyl.

The novel compounds are provided by chemical synthesis.

For example, a method for producing compounds of general formula (I), comprises the step of reacting a tetrazine derivative of formula (II) with a lysine derivative of formula (III)

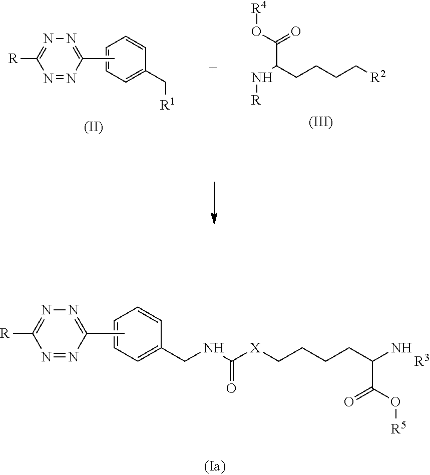

in order to achieve the intermediate compound (la), wherein
X denotes N or O,
R is selected from the group consisting of halogen, —OR^{a}, —C(O)R^{a}, —COOR^{a}, —NR^{a}R^{a}, —SR^{a}, —C₁-C₆alkyl and phenyl, wherein the —C₁-C₆alkyl or phenyl moiety is optionally substituted by halogen, —OR^{a}, —C(O)R^{a}, —COOR^{a}, —NR^{a}R^{a}, —SR^{a},
R¹ is —NH₂ or —OCN,
R² is —OH or —NH—C(═O)-imidazol,
R³ is a protecting group,
R⁴ is H or a protecting group and
R⁵ is —OH or —OCH₃, and
R^{a} is hydrogen or C₁₋₆alkyl

A protecting group or protective group is introduced into a molecule by chemical modification of a functional group to obtain chemoselectivity in a subsequent chemical reaction. Suitable protecting groups for amines are for example selected from the group consisting of carbobenzyloxy (Cbz) group, p-methoxybenzyl carbonyl (Moz or MeOZ) group, tert-butyloxycarbonyl (BOC) group, 9-fluorenylmethyloxycarbonyl (Fmoc) group, acetyl (Ac), benzoyl (Bz) group, benzyl (Bn) group, carbamate group, p-methoxybenzyl (PMB), 3,4-dimethoxybenzyl (DMPM), p-methoxyphenyl (PMP) group, tosyl (Ts) group, and Troc (trichloroethyl chloroformate) group. In one embodiment of the invention, the protecting group is a tert-butyloxycarbonyl (BOC) group.

A further embodiment of the invention relates to a peptide or protein comprising a single amino acid or multiple amino acids bearing said tetrazine moiety at predefined sites. Having amino acids bearing a tetrazine moiety at predefined sites provides the ability to produce a precisely defined peptide or protein conjugate. Having amino acids bearing a tetrazine moiety avoids problems of undefined, random labelling or incomplete labelling (if a reaction does not go to completion, heterogeneous products can result which can be a problem which is usefully addressed by synthetic amino acids bearing a tetrazine moiety).

Some embodiments of the invention relate to methods of producing a peptide or protein comprising a single or multiple tetrazine moieties, said methods comprising genetically incorporating a synthetic amino acid comprising a tetrazine moiety into a peptide or protein. Genetically incorporating the tetrazine moiety allows precise construction of a defined peptide or protein conjugate. The location of the tetrazine moieties can be precisely controlled. This advantageously avoids the need to subject the whole peptide or protein to complex reaction steps using chemical functional groups occurring in the natural amino acids.

Suitably the method described for producing the peptide or protein comprises
(i) providing a nucleic acid encoding the peptide or protein which nucleic acid comprises an orthogonal codon encoding the amino acids having a tetrazine moiety;
(ii) translating said nucleic acid in the presence of an orthogonal tRNA synthetase/tRNA pair capable of recognizing said orthogonal codon and incorporating said amino acid having a tetrazine moiety into the peptide or protein chain. Suitably said orthogonal codon comprises an amber codon (TAG), said tRNA comprises tRNAcuA and said tRNA synthetase comprises PyIRS from the organisms Methanosarcina mazei/Methanosarcina Bakeri/Methanomethylophilus alvus

Suitably said compounds are listed in Table 1.

TABLE 1

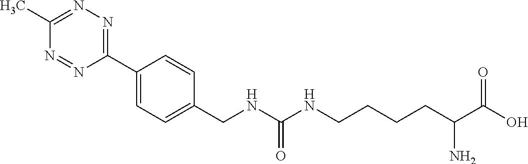 [SMILES: Cc1nnc(-c2ccc(CNC(=O)NCCCCC(N)C(=O)O)cc2)nn1]

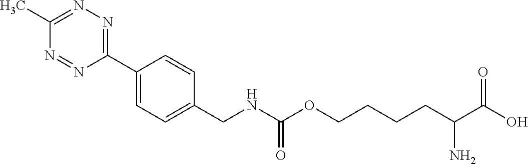 [SMILES: Cc1nnc(-c2ccc(CNC(=O)OCCCCC(N)C(=O)O)cc2)nn1]

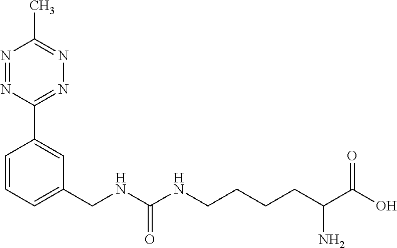 [SMILES: Cc1nnc(-c2cccc(CNC(=O)NCCCCC(N)C(=O)O)c2)nn1]

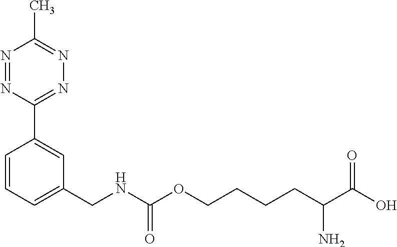 [SMILES: Cc1nnc(-c2cccc(CNC(=O)OCCCCC(N)C(=O)O)c2)nn1]

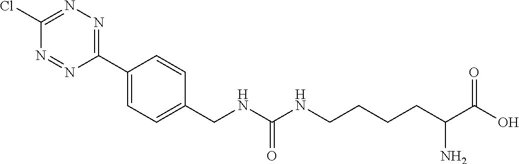 [SMILES: NC(CCCCNC(=O)NCc1ccc(-c2nnc(Cl)nn2)cc1)C(=O)O]

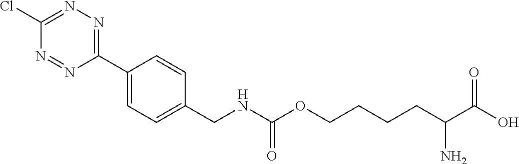 [SMILES: NC(CCCCOC(=O)NCc1ccc(-c2nnc(Cl)nn2)cc1)C(=O)O]

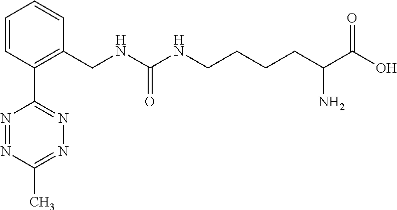 [SMILES: Cc1nnc(-c2ccccc2CNC(=O)NCCCCC(N)C(=O)O)nn1]

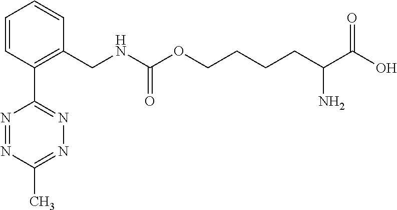 [SMILES: Cc1nnc(-c2ccccc2CNC(=O)OCCCCC(N)C(=O)O)nn1]

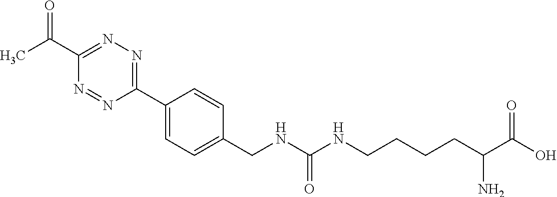 [SMILES: CC(=O)c1nnc(-c2ccc(CNC(=O)NCCCCC(N)C(=O)O)cc2)nn1]

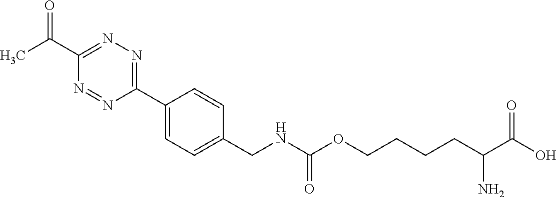 [SMILES: CC(=O)c1nnc(-c2ccc(CNC(=O)OCCCCC(N)C(=O)O)cc2)nn1]

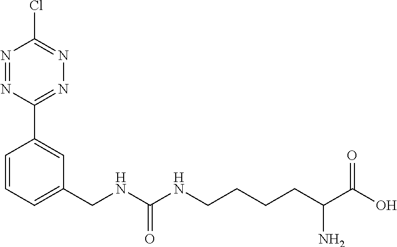 [SMILES: NC(CCCCNC(=O)NCc1cccc(-c2nnc(Cl)nn2)c1)C(=O)O]

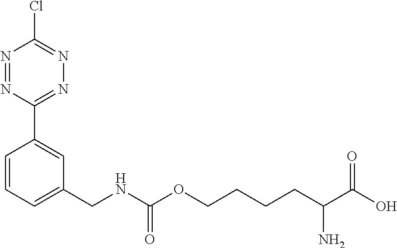 [SMILES: NC(CCCCOC(=O)NCc1cccc(-c2nnc(Cl)nn2)c1)C(=O)O]

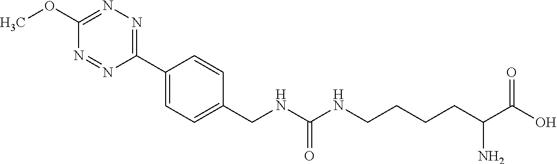 [SMILES: COc1nnc(-c2ccc(CNC(=O)NCCCCC(N)C(=O)O)cc2)nn1]

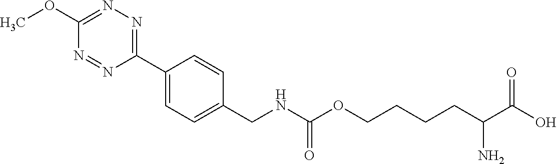 [SMILES: COc1nnc(-c2ccc(CNC(=O)OCCCCC(N)C(=O)O)cc2)nn1]

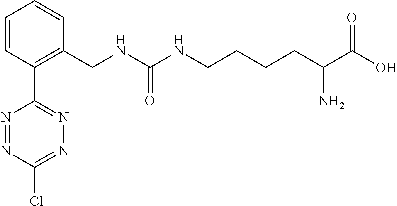 [SMILES: NC(CCCCNC(=O)NCc1ccccc1-c1nnc(Cl)nn1)C(=O)O]

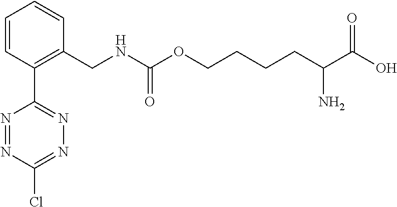 [SMILES: NC(CCCCOC(=O)NCc1ccccc1-c1nnc(Cl)nn1)C(=O)O]

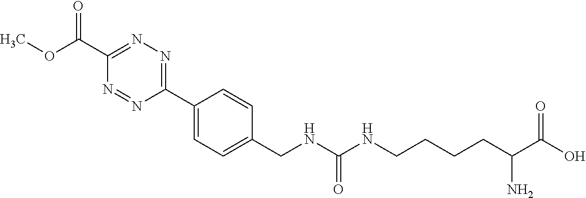 [SMILES: COC(=O)c1nnc(-c2ccc(CNC(=O)NCCCCC(N)C(=O)O)cc2)nn1]

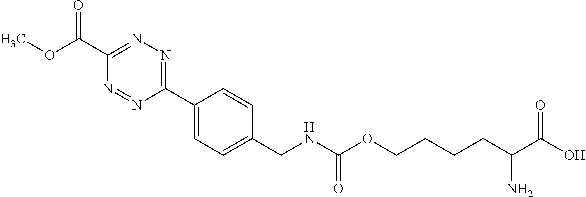 [SMILES: COC(=O)c1nnc(-c2ccc(CNC(=O)OCCCCC(N)C(=O)O)cc2)nn1]

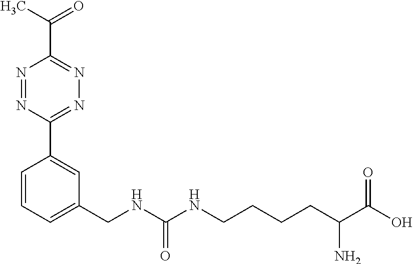 [SMILES: CC(=O)c1nnc(-c2cccc(CNC(=O)NCCCCC(N)C(=O)O)c2)nn1]

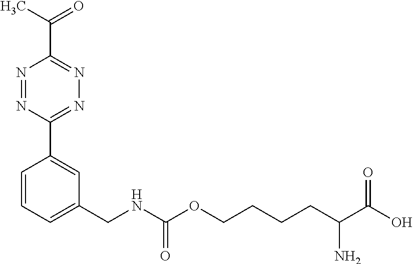 [SMILES: CC(=O)c1nnc(-c2cccc(CNC(=O)OCCCCC(N)C(=O)O)c2)nn1]

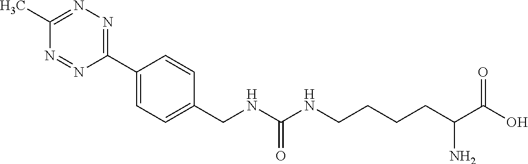 [SMILES: Cc1nnc(-c2ccc(CNC(=O)NCCCCC(N)C(=O)O)cc2)nn1]

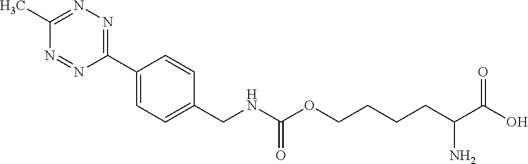 [SMILES: Cc1nnc(-c2ccc(CNC(=O)OCCCCC(N)C(=O)O)cc2)nn1]

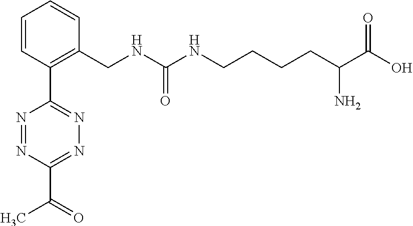 [SMILES: CC(=O)c1nnc(-c2ccccc2CNC(=O)NCCCCC(N)C(=O)O)nn1]

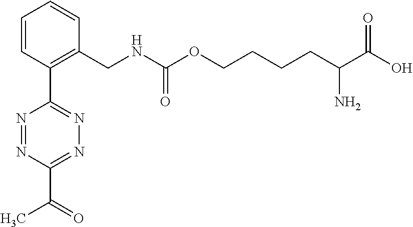 [SMILES: CC(=O)c1nnc(-c2ccccc2CNC(=O)OCCCCC(N)C(=O)O)nn1]

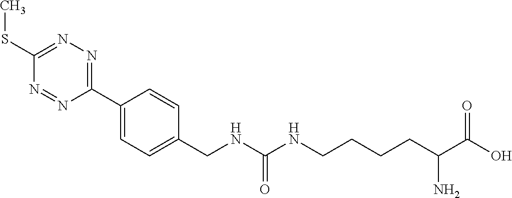 [SMILES: CSc1nnc(-c2ccc(CNC(=O)NCCCCC(N)C(=O)O)cc2)nn1]

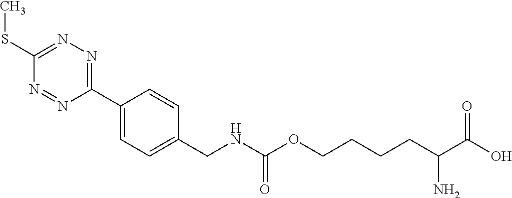 [SMILES: CSc1nnc(-c2ccc(CNC(=O)OCCCCC(N)C(=O)O)cc2)nn1]

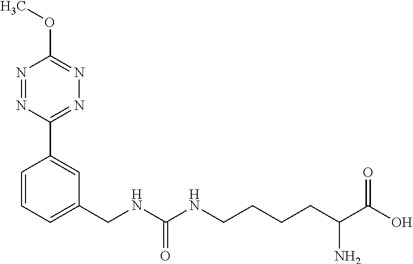 [SMILES: COc1nnc(-c2cccc(CNC(=O)NCCCCC(N)C(=O)O)c2)nn1]

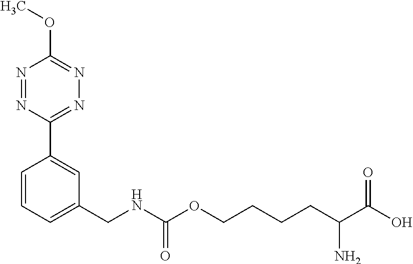 [SMILES: COc1nnc(-c2cccc(CNC(=O)OCCCCC(N)C(=O)O)c2)nn1]

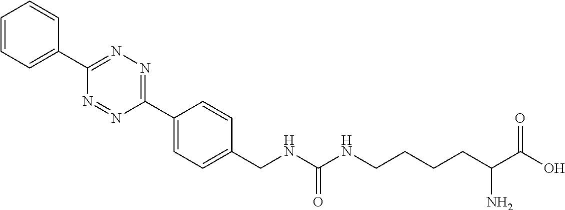 [SMILES: NC(CCCCNC(=O)NCc1ccc(-c2nnc(-c3ccccc3)nn2)cc1)C(=O)O]

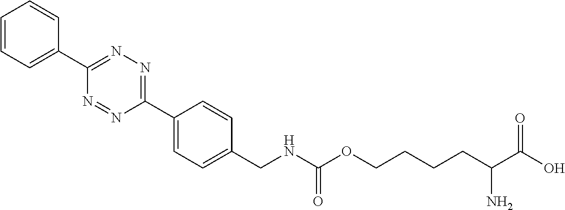 [SMILES: NC(CCCCOC(=O)NCc1ccc(-c2nnc(-c3ccccc3)nn2)cc1)C(=O)O]

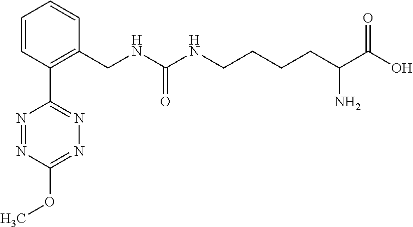 [SMILES: COc1nnc(-c2ccccc2CNC(=O)NCCCCC(N)C(=O)O)nn1]

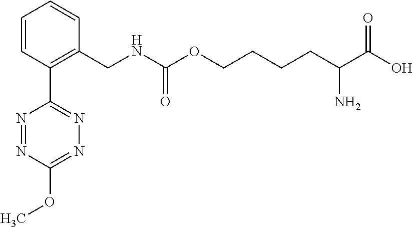 [SMILES: COc1nnc(-c2ccccc2CNC(=O)OCCCCC(N)C(=O)O)nn1]

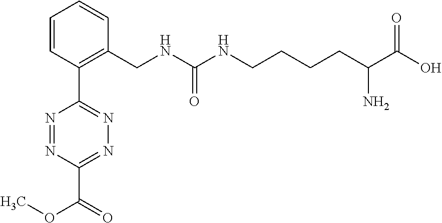 [SMILES: COC(=O)c1nnc(-c2ccccc2CNC(=O)NCCCCC(N)C(=O)O)nn1]

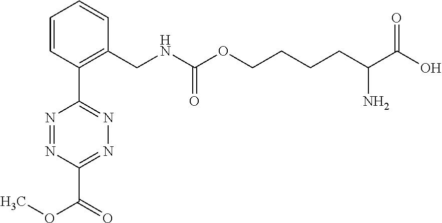 [SMILES: COC(=O)c1nnc(-c2ccccc2CNC(=O)OCCCCC(N)C(=O)O)nn1]

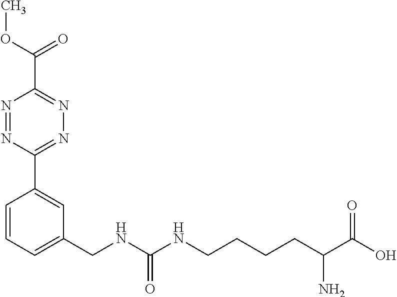 [SMILES: COC(=O)c1nnc(-c2cccc(CNC(=O)NCCCCC(N)C(=O)O)c2)nn1]

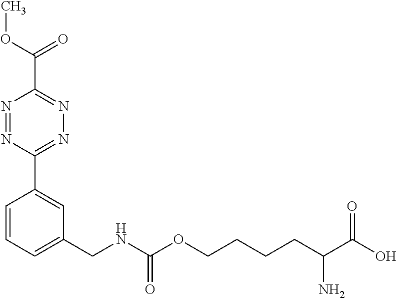 [SMILES: COC(=O)c1nnc(-c2cccc(CNC(=O)OCCCCC(N)C(=O)O)c2)nn1]

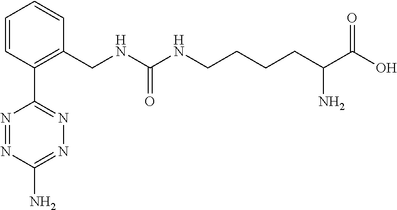 [SMILES: Nc1nnc(-c2ccccc2CNC(=O)NCCCCC(N)C(=O)O)nn1]

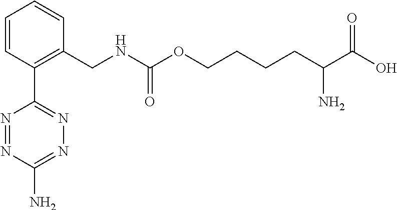 [SMILES: Nc1nnc(-c2ccccc2CNC(=O)OCCCCC(N)C(=O)O)nn1]

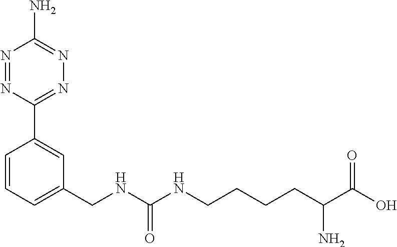 [SMILES: Nc1nnc(-c2cccc(CNC(=O)NCCCCC(N)C(=O)O)c2)nn1]

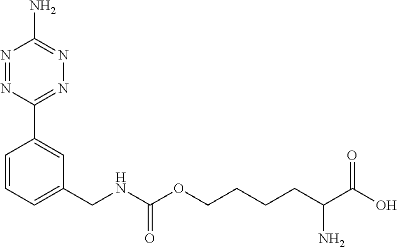 [SMILES: Nc1nnc(-c2cccc(CNC(=O)OCCCCC(N)C(=O)O)c2)nn1]

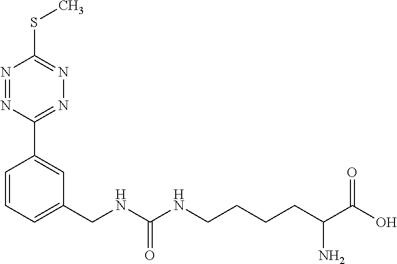 [SMILES: CSc1nnc(-c2cccc(CNC(=O)NCCCCC(N)C(=O)O)c2)nn1]

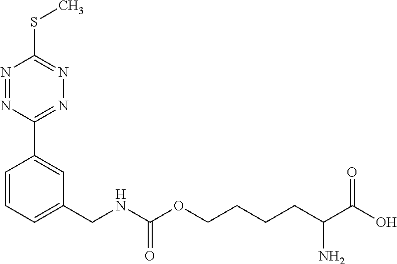 [SMILES: CSc1nnc(-c2cccc(CNC(=O)OCCCCC(N)C(=O)O)c2)nn1]

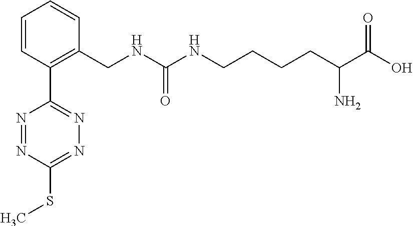 [SMILES: CSc1nnc(-c2ccccc2CNC(=O)NCCCCC(N)C(=O)O)nn1]

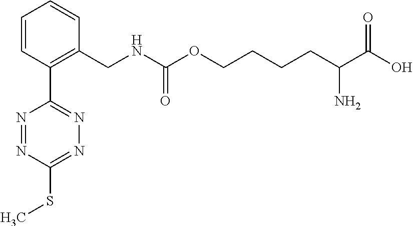 [SMILES: CSc1nnc(-c2ccccc2CNC(=O)OCCCCC(N)C(=O)O)nn1]

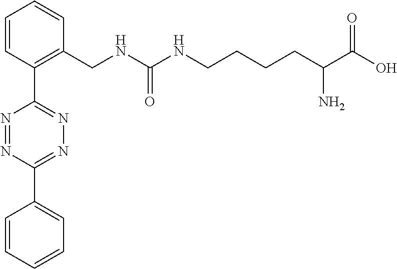 [SMILES: NC(CCCCNC(=O)NCc1ccccc1-c1nnc(-c2ccccc2)nn1)C(=O)O]

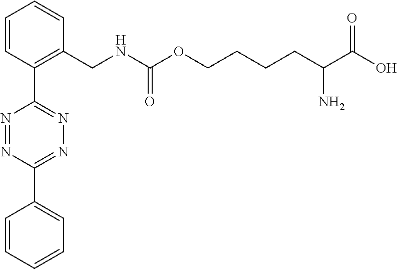 [SMILES: NC(CCCCOC(=O)NCc1ccccc1-c1nnc(-c2ccccc2)nn1)C(=O)O]

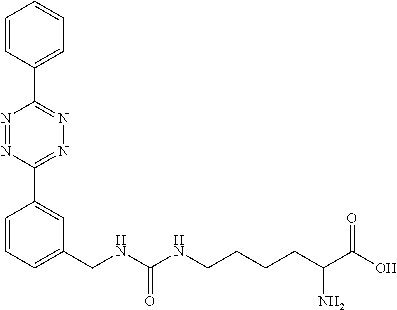 [SMILES: NC(CCCCNC(=O)NCc1cccc(-c2nnc(-c3ccccc3)nn2)c1)C(=O)O]

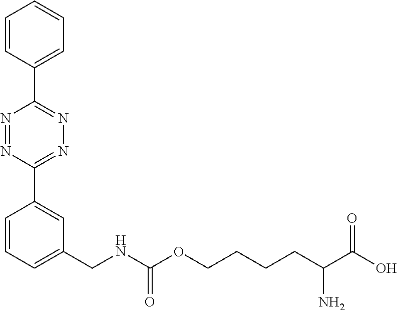 [SMILES: NC(CCCCOC(=O)NCc1cccc(-c2nnc(-c3ccccc3)nn2)c1)C(=O)O]

In some embodiments the peptide or protein comprises a single tetrazine moiety. This has the advantage of providing specificity for any further chemical modifications which might be directed at the tetrazine moiety. For example when there is only a single tetrazine moiety in the peptide or protein of interest then possible issues of partial modification (e.g., where only a subset of tetrazine moieties in the peptide or protein are subsequently modified), or issues of reaction microenvironments varying between alternate tetrazine moieties in the same peptide or proteins (which could lead to unequal reactivity between different tetrazine moiety(s) at different locations in the peptide or protein) are avoided. Therefore, in some embodiments the peptide or protein comprises a single tetrazine residue.

A key advantage of incorporation of tetrazine moiety is that is permits a range of extremely useful further compounds such as labels or pharmaceutically active substances to be easily and specifically attached to the tetrazine moiety.

The compounds and methods described herein include the use of Diels-Alder pairs that include a diene and a dienophile. The inverse electron demand Diels-Alder cycloaddition reaction of a diene (e.g., a tetrazine) with a dienophile (e.g., an alkene or alkyne), produces an unstable cycloadduct which subsequently undergoes a retro-Diels-Alder cycloaddition reaction to produce dinitrogen as a byproduct and the desired dihydropyrazine (after reaction with an alkene) or pyrazine (after reaction with an alkyne) products. The dihydropyrazine product may undergo an additional oxidation step to generate the corresponding pyrazine. The dienophile may be a chemical moiety which preferably does not contain a terminal double bond. For example, the dienophile is a cyclopropene, alkene, norbornadiene, azonorbornadiene, oxonorbornadiene, trans-cyclooctene, norbornene, or a vinyl ether. A further embodiment of the invention relates to said tetrazine moiety which is linked to an electron-rich dienophile compound or a strained dienophile compound. Strained dienophiles are for example norbornene and trans-cyclooctene.

The electron-rich dienophile compound or a strained dienophile compound may further be joined to a fluorophore or to a PEG group or to a pharmaceutically active substance or to another protein or peptide or sugar polymers or a solid surface

In principle the invention can be applied to any position in the peptide or protein. Suitably the invention is not applied to the N-terminal amino acid of the peptide or protein. When selecting the position of the amino acid to be targeted in the peptide or protein of interest, it is advantageous to select a surface residue. Surface residues may be determined by sequence analysis, or by three-dimensional molecular modelling. Surface residues may be determined by any suitable method known in the art. Advantages of targeting surface residues include better presentation of dyes such as fluorophores or labels such as biophysical labels. Advantages of targeting surface residues include simpler or more efficient downstream modifications. Advantages of targeting surface residues include less likelihood of disruption of peptide or protein structure and/or function by application of the label.

Particularly suitable amino acid residues to target in the peptide or protein of interest include non-hydrophobic residues, e.g., hydrophilic residues or polar residues. Hydrophobic residues are less preferably targeted according to the invention. Amino acids such as glycine, alanine, serine, isoleucine, leucine, threonine, glutamic acid, proline, methionine, arginine, asparagine, glutamine, lysine, or cysteine are suitably targeted. Preferably, glycine, alanine, serine, or lysine are suitable targets. “Targeted” as used herein means substituting the codon for the residue being targeted for the orthogonal codon and synthesizing the peptide or protein as described herein.

In another aspect, the invention relates to a homogenous recombinant peptide or protein as described above. Suitably said peptide or protein is made by a method as described above.

A further embodiment of the invention relates to a peptide or protein produced according to the method(s) described herein. As well as being the product of those new methods, such a peptide or protein has the advantageous technical feature of comprising a tetrazine moiety.

Mutating has its normal meaning in the art and may refer to the substitution or truncation or deletion of the residue, motif or domain referred to. Mutation may be effected at the peptide or protein level e.g., by synthesis of a peptide or protein having the mutated sequence, or may be effected at the nucleotide level e.g., by making a nucleic acid encoding the mutated sequence, which nucleic acid may be subsequently translated to produce the mutated peptide or protein. Where no amino acid is specified as the replacement amino acid for a given mutation site, suitably a randomization of said site may be used.

A fragment is at least 10 amino acids in length, or at least 25 amino acids, or at least 50 amino acids, or at least 100 amino acids, or at least 200 amino acids, or at least 250 amino acids, or at least 300 amino acids, or the majority of the peptide or protein of interest.

In the method according to the invention, said genetic incorporation preferably uses an orthogonal or expanded genetic code, in which one or more specific orthogonal codons have been allocated to encode the specific amino acid residue with the tetrazine moiety so that it can be genetically incorporated by using an orthogonal tRNA synthetase/tRNA pair. The orthogonal tRNA synthetase/tRNA pair can in principle be any such pair capable of charging the tRNA with the amino acid comprising the tetrazine moiety and capable of incorporating that amino acids comprising the tetrazine moiety into the peptide or protein chain in response to the orthogonal codon. The orthogonal codon may be the orthogonal codon amber, ochre, opal or a quadruplet codon or any other triplet codon. The codon simply has to correspond to the orthogonal tRNA which will be used to carry the amino acid comprising the tetrazine moiety. Preferably the orthogonal codon is amber.

Polynucleotides encoding the peptide or protein of interest for the method as described herein may be incorporated into a recombinant replicable vector. The vector may be used to replicate the nucleic acid in a compatible host cell. Thus in a further embodiment, the invention provides a method of making polynucleotides of the invention by introducing a polynucleotide according to the invention into a replicable vector, introducing the vector into a compatible host cell, and growing the host cell under conditions which allow replication of the vector. The vector may be recovered from the host cell. Suitable host cells include bacteria such as E. coli as well as yeasts such as S. cerevisiae and P. pastoris as well as higher eukaryotic host cells like insect cells, HEK cells and Chinese Hamster Ovary cells.

Preferably, a polynucleotide of the invention in a vector is operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences. Vectors of the invention may be transformed or transfected into a suitable host cell as described to provide for expression of a protein of the invention. This process may comprise culturing a host cell transformed with an expression vector as described above under conditions to provide for expression by the vector of a coding sequence encoding the protein, and optionally recovering the expressed protein.

The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid. Vectors may be used, for example, to transfect or transform a host cell.

Control sequences operably linked to sequences encoding the protein of the invention include promoters/enhancers and other expression regulation signals. These control sequences may be selected to be compatible with the host cell for which the expression vector is designed to be used in. The term promoter is well-known in the art and encompasses nucleic acid regions ranging in size and complexity from minimal promoters to promoters including upstream elements and enhancers.

Another aspect of the invention is a method, such as an in vivo method, of incorporating the tetrazine containing amino acid(s) genetically and site-specifically into the protein of choice, suitably in a bacterial or eukaryotic cell. One advantage of incorporating genetically by said method is that it obviates the need to deliver the proteins comprising the tetrazine amino acid into a cell once formed, since in this embodiment they may be synthesized directly in the target cell. The method comprises the following steps:
i) introducing, or replacing a specific codon with, an orthogonal codon such as an amber codon at the desired site in the nucleotide sequence encoding the protein
ii) introducing an expression system of orthogonal tRNA synthetase/tRNA pair in the cell, such as an engineered pyrrolysyl-tRNA synthetase/tRNA pair
iii) growing the cells in a medium with the tetrazine containing amino acid according to the invention.

Step (i) entails or replacing a specific codon with an orthogonal codon such as an amber codon at the desired site in the genetic sequence of the protein. This can be achieved by simply introducing a construct, such as a plasmid, with the nucleotide sequence encoding the protein, wherein the site where the tetrazine containing amino acid is desired to be introduced/replaced is altered to comprise an orthogonal codon such as an amber codon. This is well within the skilled person's ability and examples of such are given herein.

Step (ii) requires an orthogonal expression system to specifically incorporate the tetrazine containing amino acid at the desired location (e.g., the amber codon). Thus a specific orthogonal tRNA synthetase such as an orthogonal engineered pyrrolysyl-tRNA synthetase and a specific corresponding orthogonal tRNA pair which are together capable of charging said tRNA with the tetrazine containing amino acid are required. Examples of these are provided herein.

Host cells comprising polynucleotides according to the invention may be used to express proteins of the invention. Host cells may be cultured under suitable conditions which allow expression of the proteins of the invention. Expression of the proteins of the invention may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Peptides or proteins of the invention can be extracted from host cells by a variety of techniques known in the art, including enzymatic, chemical and/or osmotic lysis and physical disruption.

Peptides or proteins of the invention can be purified by standard techniques known in the art such as preparative chromatography, affinity purification or any other suitable technique.

Suitably the tetrazine moiety incorporated into the peptide or protein of interest is reacted with an electron-rich dienophile or a strained dienophile compound. The electron-rich dienophile or a strained dienophile compound acts to conveniently attach a molecule of interest to the peptide or protein via the tetrazine moiety. Thus, the electron-rich dienophile or a strained dienophile compound may already bear the molecule of interest.

Suitably said electron-rich dienophile or a strained dienophile compound may be further joined to any suitable molecule of interest for attaching same to the peptide or protein via the tetrazine reaction.

The tetrazine containing peptide or protein of the invention may be conveniently conjugated to other biophysical labels than fluorophores, for example, NMR probes, Spin label probes, IR labels, EM-probes as well as small molecules, oligonucleotides, lipids, nanoparticles, quantum dots, biophysical probes (EPR labels, NMR labels, IR labels), small molecules (biotin, drugs, lipids), oligonucleotides (DNA, RNA, LNA, PNA), particles (nanoparticles, viruses), polymers (PEG, PVC), proteins, peptides, surfaces and the like.

The novel amino acids bearing a tetrazine moiety are specifically useful for incorporation into peptides or proteins. Thus conjugation of the peptide or protein to moieties bearing an electron-rich dienophile or strained dienophile moiety is envisaged. The modified peptide or proteins may be used as building blocks for active pharmaceutical ingredients. The novel amino acid compounds having the tetrazine moiety may be incredibly useful as building blocks in peptide chemistry and as novel synthons for pharmaceutical ingredients.

The compounds are specifically useful as a building block for the chemical or enzymatic synthesis of peptide or proteins, or analogues or precursors thereof. It is understood that the term “building block” refers to structural units which are used in chemical or enzymatic operations.

In the context of the present invention, the term “synthon” refers to a compound that is, or can be used as, a synthetic equivalent for a particular compound of interest in a chemical reaction, e.g. in the synthesis of an active pharmaceutical ingredient.

### Methods

### Synthesis of an Amino Acid Compound Linked Via a Urea Group to a Tetrazine Moiety.

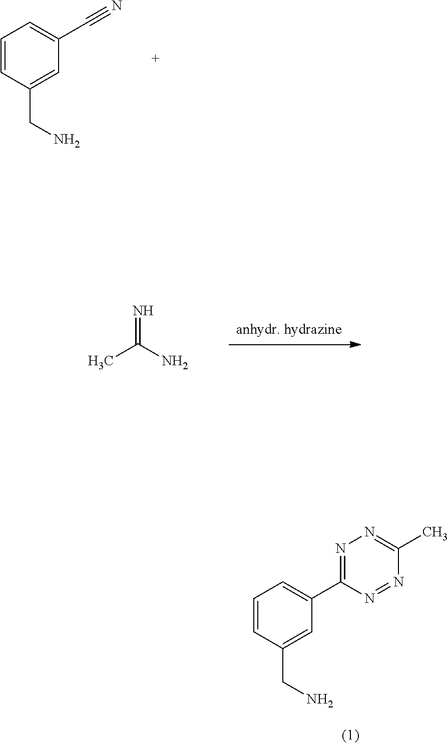

Synthesized by mixing 2 mmol 3-(aminomethyl) benzonitrile with 10 mmol acetamidine hydrochloride under N₂. Anhydrous hydrazine (2 mL) was then added slowly to the solid mixture with stirring. The reactions mixture was then stirred at room temperature or with heat for 30 min to 2 h. Sodium nitrite (10 mmol) in water was added to the reaction mixture followed by dropwise addition of 2% aqueous HCl until the solution reached approximately pH 3. The solution turned red and stopped bubbling indicating that the dihydrotetrazines were oxidized to the tetrazines.

The oxidized acidic solution was extracted with dichloromethane (DCM) until the organic layer was colorless. The organic fractions were discarded and the aqueous layer was then saturated with NaCl and basified by addition of solid NaHCO₃ and immediately extracted with DCM. The organic layers were then dried with MgSO₄, filtered, and the solvent was removed by rotary evaporation to yield a crude product mixture comprising 1-[3-(6-methyl-1,2,4,5-tetrazin-3-yl)phenyl]methanamine (Karver M. R., et al. (2011).

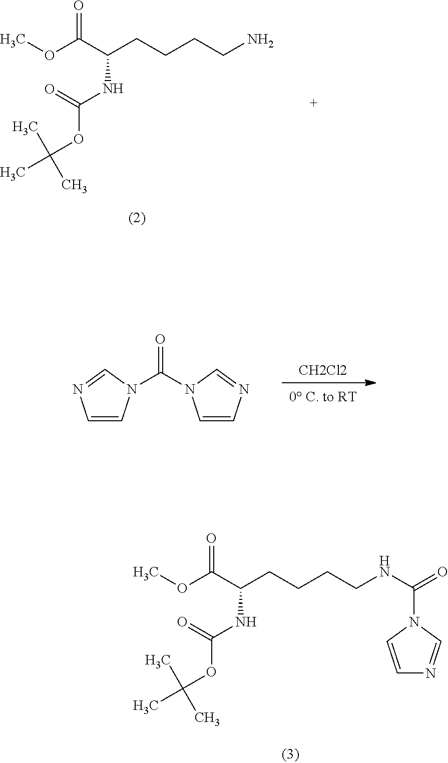

A solution of Boc-Lys-OMe ((2), 3.3 g, 12.7 mmol) in 65 mL dry CH₂Cl₂ was added into a solution of 1,1′-carbonyl-di-imidazole (4 g, 24.8 mmol) in 20 mL dry CH₂Cl₂ dropwise over 1 h at 0° C. The temperature was allowed to rise to room temperature gradually after the addition of Boc-Lys-OMe and the solutions was stirred overnight at room temperature. The reaction was monitored by 12 or KMnO₄ on TLC (Thin Layer Chromotography) and the reaction solution was washed with brine. The aqueous phase was extracted with CH₂Cl₂ twice, while the organic phase was combined and dried over Na₂SO₄, evaporated under vacuum, and the product (3) was eluted in flash chromatography by CH₂Cl₂/MeOH (25:1) with a yield of 85%.

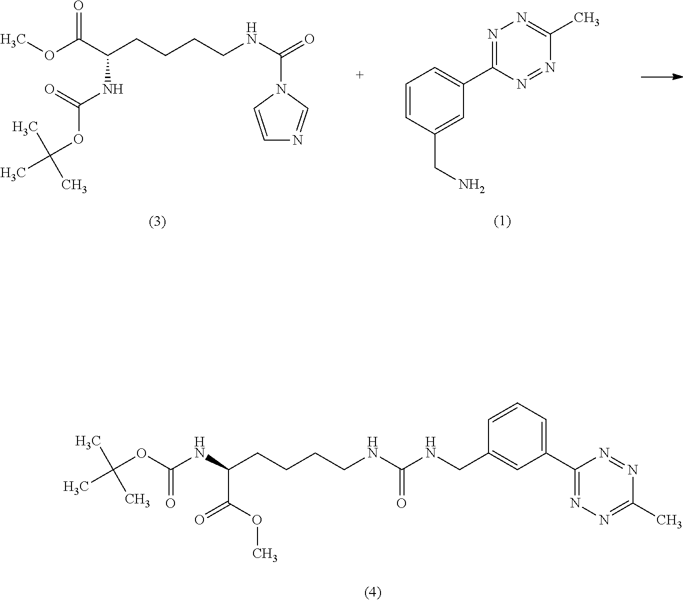 [SMILES: COC(=O)[C@H](CCCCNC(=O)n1ccnc1)NC(=O)OC(C)(C)C ; Cc1nnc(-c2cccc(CN)c2)nn1 ; COC(=O)[C@H](CCCCNC(=O)NCc1cccc(-c2nnc(C)nn2)c1)NC(=O)OC(C)(C)C]

Activated lysine-derivative ((3), 1.9 g, 5.4 mmol) and tetrazine-amine (1.19 g, 5.9 mmol) was mixed in 30 mL dry CH₃CN, and the reaction was stirred at 50° C. for 24 h, followed by solvent evaporation. The product was dissolved in 80 ml CH₂Cl₂. The organic phase was washed with 30 mL 1 M HCl and 20 mL saturated NaHCO₃, dried over Na₂SO₄, and evaporated under vacuum. The final product (4) was eluted by flash chromatography with CH₂Cl₂/MeOH (30:1) (Zhang M. et al., (2011)).

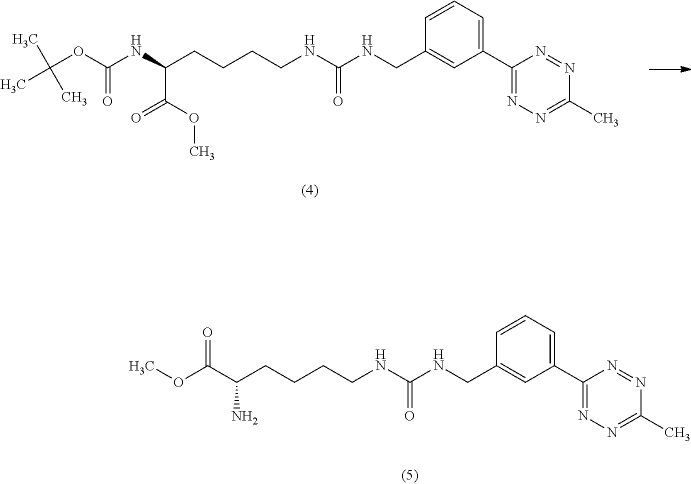

The desired compound (5) was obtained via standard Boc-deprotection using TFA.

### Synthesis of an Amino Acid Compound Linked Via a Carbamate Group to a Tetrazine Moiety.

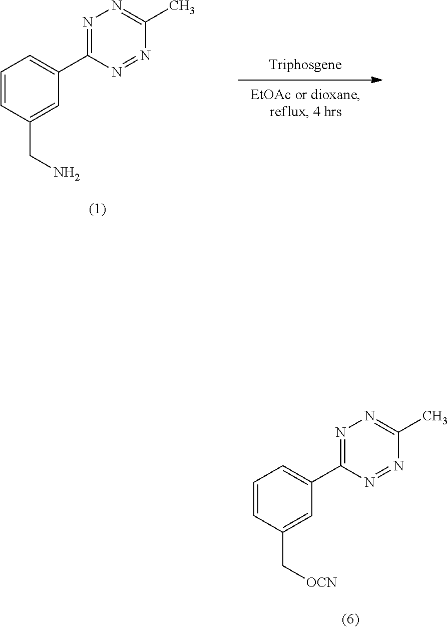

1-[3-(6-methyl-1,2,4,5-tetrazin-3-yl)phenyl]methanamine (1) was obtained as described above. The further synthesis is analogous to the process as described by Charalambides Y. C. and Moratti S. C. (2007). To a solution of triphosgene (2.52 g, 8.50 mmol, 0.5 equiv) in EtOAc (100 mL), 1-[3-(6-methyl-1,2,4,5-tetrazin-3-yl) phenyl]methanamine ((1), 17.00 mmol) in EtOAc (40 mL), was added in small portions. The mixture was then refluxed under nitrogen for 4 h. After allowing the reaction to cool to room temperature, the solvent was evaporated under reduced pressure and the residue obtained was subjected to distillation in a Kugelrohr apparatus, affording product ((6), (87%)) as a light-yellow liquid

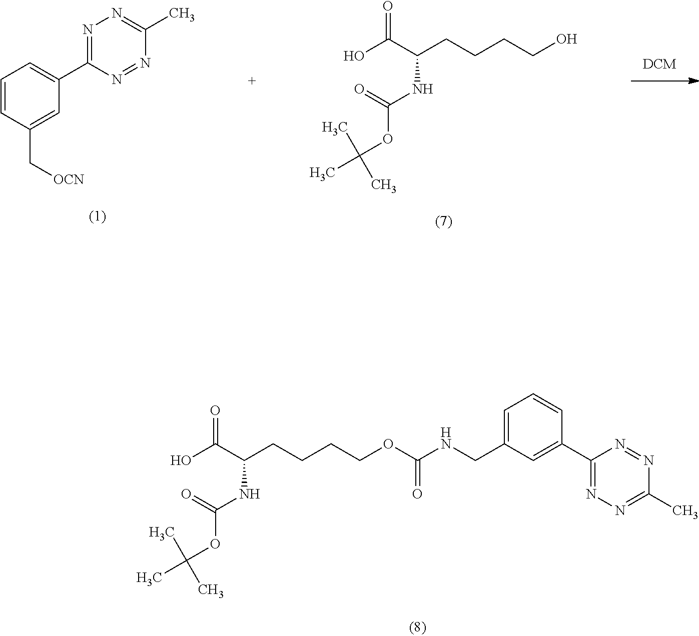

The intermediate compound (8) was synthesized as described by Torres-Kolbus J. et al. (2014). Briefly, 6-hydroxy-Boc-L-norleucine-OH ((7), 25 mg, 0.10 mmol) was dissolved in a solution of dry DCM (1 mL) and DIPEA (53 mL, 0.30 mmol). The solution was chilled to 0° C. before the addition of tetrazine isocyanate ((1), 0.20 mmol) and the reaction was allowed to proceed at 40° C. overnight. After cooling to room temperature, the mixture was diluted with DCM (3 mL) and 5% citric acid (4 mL) was added. The aqueous layer was extracted with DCM (3×4 mL) and the combined organic layers were washed with water (10 mL) and brine (5 mL). The resulting organic layer was dried over Na₂SO₄, filtered and concentrated in vacuo to dryness to obtain product (8).

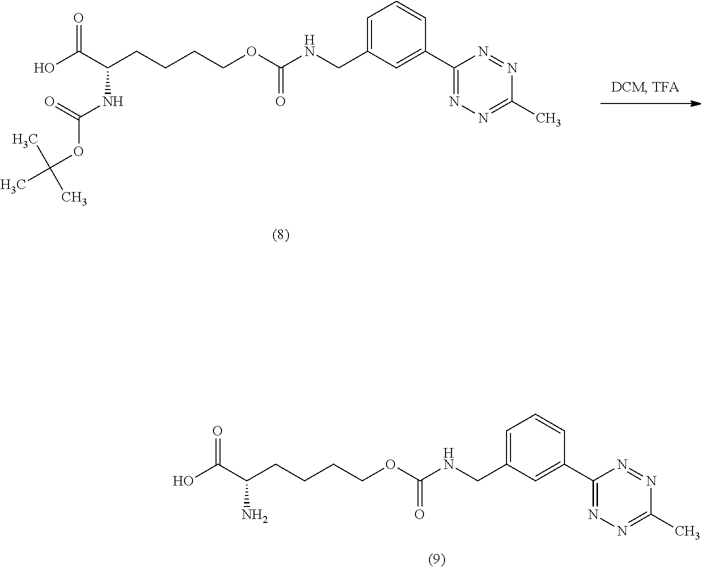 [SMILES: Cc1nnc(-c2cccc(CNC(=O)OCCCC[C@H](NC(=O)OC(C)(C)C)C(=O)O)c2)nn1 ; Cc1nnc(-c2cccc(CNC(=O)OCCCC[C@H](N)C(=O)O)c2)nn1]

The desired compound (9) was obtained via standard Boc-deprotection using TFA.

Incorporation into a Protein:

A mutant pyrrolysyl-tRNA synthetase, obtained from a wild-type pyrrolysyl-tRNA synthetase, which is Methanosarcina, Methanocaldococcus, Methanomethylophilus or other derived pyrrolysyl-tRNA synthetase, and/or the mutant pyrrolysyl-tRNA synthetase aminoacylates a pyrrolysine tRNA, incorporates amino acids as described herein.

Incorporation of the Tetrazine Amino Acid into Red Fluorescent Protein (RFP)

A mutant pyrrolysyl-tRNA synthetase, obtained from a wild-type pyrrolysyl-tRNA synthetase, which is an archaeal-derived pyrrolysyl-tRNA synthetase (such as Methanosarcina or Methanocaldococcus or Methanomethylophilus or other), and/or the mutant pyrrolysyl-tRNA synthetase aminoacylates a pyrrolysine tRNA, incorporates amino acids as described herein. The mutant pyrrolysyl-tRNA synthetase was generated by state-of-the-art protein engineering technologies, such as structure guided site-saturation mutagenesis or directed evolution or a combination. Also, other technologies such as gene shuffling would be possible.

The mutant pyrrolysyl-tRNA synthetase and the corresponding amber suppressor pyrrolysine tRNA were introduced into an expression vector harboring a pColE1 origin of replication, a variant of the red fluorescent protein reporter carrying an in-frame amber stop codon at amino acid position 20, as well as a C-terminal hexahistidine tag and a kanamycin resistance gene. The mutant pyrrolysyl-tRNA synthetase was expressed from an arabinose inducible promoter and the suppressor pyrrolysine tRNA from a constitutive promoter commonly used for this purpose.

E. coli cells harboring the above-described expression vector were cultivated in 250 mL flasks each containing 50 mL M9 minimal medium with 1-2% glucose as C-source or standard 2×YT medium with 50 μg/mL kanamycin (Roth). Cultures were incubated at 37° C. on an orbital shaker at 160-180 rpm. At D600 of 0.8-1.0, the expression of the PyIRS was induced by adding 0.2% (w/v) of arabinose (Roth). In addition, 0.1-10 mM of tetrazine-lysine dissolved in 0.1 M HCl or DMSO or H₂O or a mixture of the previous. Expression was carried out between 4-24 hours (temperature can be adjusted depending on the target protein; 37° C. for RFP). Cells were harvested by centrifugation (5,000 g for 30 minutes at 4° C.). The RFP variant was purified by Ni2+-affinity chromatography using Ni-NTA agarose following the instructions of the manufacturer.

Purified RFP variant carrying the tetrazine-lysine was modified by conjugation chemistry applying a trans-cyclooctene (TCO) bearing fluorescent dye as reaction partner, such as TCO-TAMRA. Reactions were performed in 100 mM MES Buffer pH 6 and incubated between 4-24 hours. TAMRA labeled RFP samples were separated on pre-casted SDS gel following the instructions of the manufacturer. The gels were exposed to UV-light to detect TAMRA fluorescence and subsequently stained with Coomassie Blue following standard procedures. The band at the expected size of RFP (˜28 kDa, see Scheme I) was excised.

The presence of the tetrazine-lysine compound, as well as the TAMRA modification was confirmed by peptide sequencing by tandem mass spectrometry. The successful TAMRA modification was also confirmed by obtaining a signal for TAMRA fluorescence at the size of RFP.

### Chemical Click Reactions

The compounds of general formula (I) are specifically useful in chemical click reactions.

Reaction of 3-Methyl-6-Phenyltetrazines with Dienophile and Strained Dienophile Partners.

The tetrazine moiety reacts with trans-cyclooctene, cyclopropene, norbornene, spirohexene, or styrene. The reactions are usually conducted in aqueous environments at physiological pH values of 6.5-8 in varying salt and buffer concentrations. The temperature is in the range of 0° C. to 100° C.

### Scheme I—Chemical Click Reactions

### TCO-Tetrazine Reactions

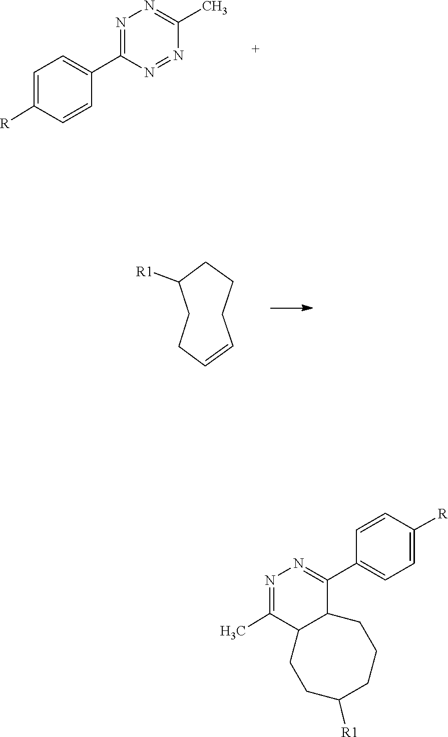

### Cyclopropene-Tetrazine Reactions

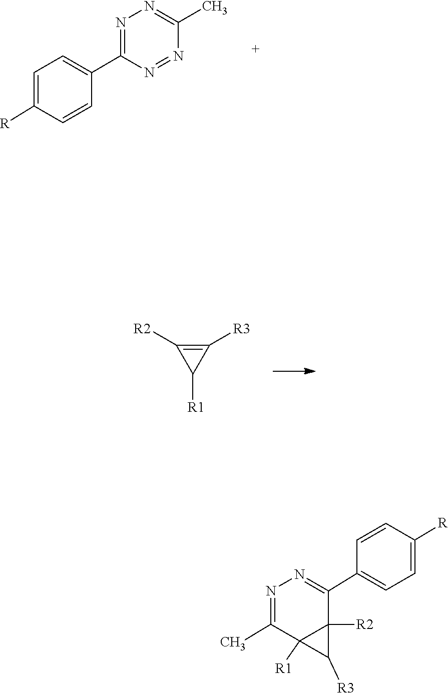

### Norbornene-Tetrazine Reactions

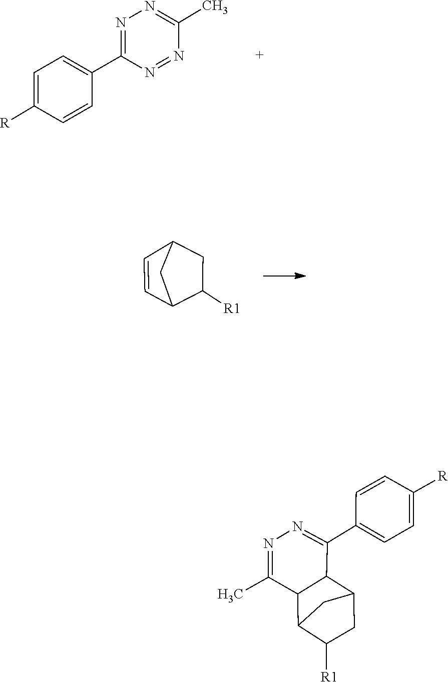

### Spirohexene-Tetrazine Reactions

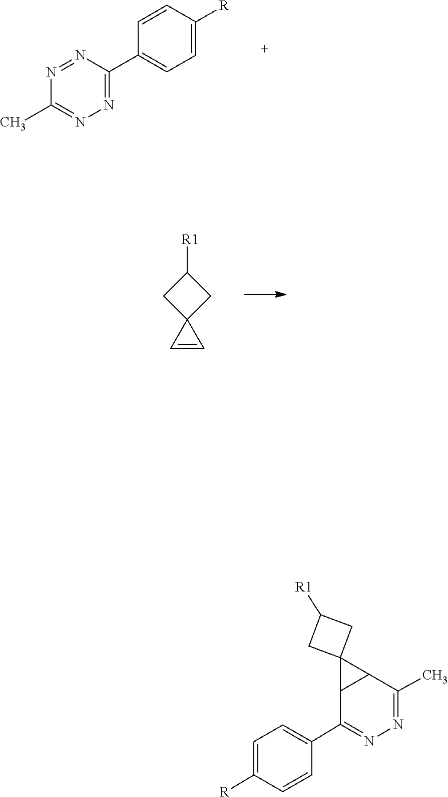

### Styrene-Tetrazine Reactions

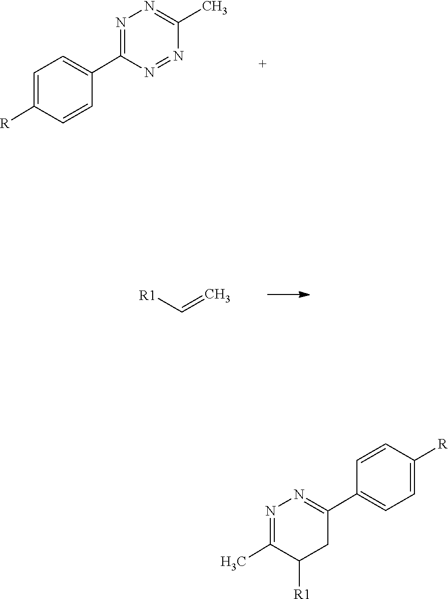

### REFERENCES

1) Hu Q.-Y. et al. Towards the next generation of biomedicines by site-selective conjugation. Chem. Soc. Rev., 2016, 45(6), 1691-1719.
2) Young, T. S. et al. An Enhanced System for Unnatural Amino Acid Mutagenesis in E. coli. Journal of Molecular Biology, 2010, 395(2), 361-374.
3) Wan, W. et al. Pyrrolysyl-tRNA synthetase: An ordinary enzyme but an outstanding genetic code expansion tool. Biochimica et Biophysica Acta—Proteins and Proteomics 2014, Vol. 1844, Issue 6, pp. 1059-1070).
4) Owens A. E., et al. Two-Tier Screening Platform for Directed Evolution of Aminoacyl-tRNA Synthetases with Enhanced Stop Codon Suppression Efficiency. ChemBioChem 2017, 18(12), 1109-1116.
5) Yanagisawa T. et al. Structural Basis for Genetic-Code Expansion with Various Bulky Lysine Derivatives by an Engineered Pyrrolysyl-tRNA Synthetase. SSRN Electronic Journal 2018, 1-14.
6) Hohl A. et al. Engineering a promiscuous pyrrolysyl-{tRNA} synthetase by a high throughput {FACS} screen. BioRxiv 2017, 229054.
7) Lang K., and Chin, J. W. Bioorthogonal Reactions for Labeling Proteins. ACS Chemical Biology 2014, 9(1), 16-20.
8) Eising S. et al. Highly Stable and Selective Tetrazines for the Coordination-Assisted Bioorthogonal Ligation with Vinylboronic Acids. Bioconjugate Chemistry 2018, 29(9), 3054-3059.
9) Zeglis B. M. et al. Building blocks for the construction of bioorthogonally reactive peptides via solid-phase peptide synthesis. ChemistryOpen 2014, 3(2), 48-53.
10) Mayer S. V. et al. Photo-induced and Rapid Labeling of Tetrazine-Bearing Proteins via Cyclopropenone-Caged Bicyclononynes. Angewandte Chemie-International Edition 2019, 58(44), 15876-15882.
11) Karver M. R. et al. Synthesis and Evaluation of a Series of 1,2,4,5-Tetrazines for Bioorthogonal Conjugation. Bioconjugate Chemistry 2011, 22 (11), 2263-2270.
12) Zhang M. et al. A genetically incorporated crosslinker reveals chaperone cooperation in acid resistance. Nat Chem Biol 2011, 7, 671-677.
13) Charalambides Y. C. and Moratti S. C. Comparison of Base—Promoted and Self—Catalyzed Conditions in the Synthesis of Isocyanates from Amines Using Triphosgene, Synthetic Communications 2007, 37:6, 1037-1044.
14) Torres-Kolbus J. et al. Synthesis of Non-linear Protein Dimers through a Genetically Encoded Thiol-ene Reaction. PLOS ONE 2014, 9(9): e105467.

1. A compound of general formula (I), 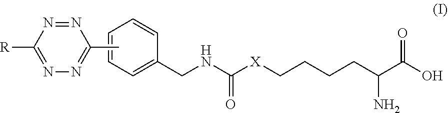
wherein
X denotes N or O, and
R is selected from the group consisting of halogen, —OR^{a}, —C(O)R^{a}, —COOR^{a}, —NR^{a}R^{a}, —SR^{a}, —C₁₋₆alkyl and phenyl, wherein the —C₁₋₆alkyl or phenyl moiety is optionally substituted by halogen, —OR^{a}, —C(O)R^{a}, —COOR^{a}, —NR^{a}R^{a}, —SR^{a}, and R^{a} is hydrogen or C₁₋₆alkyl.

2. The compound according to claim 1, selected from the group consisting of

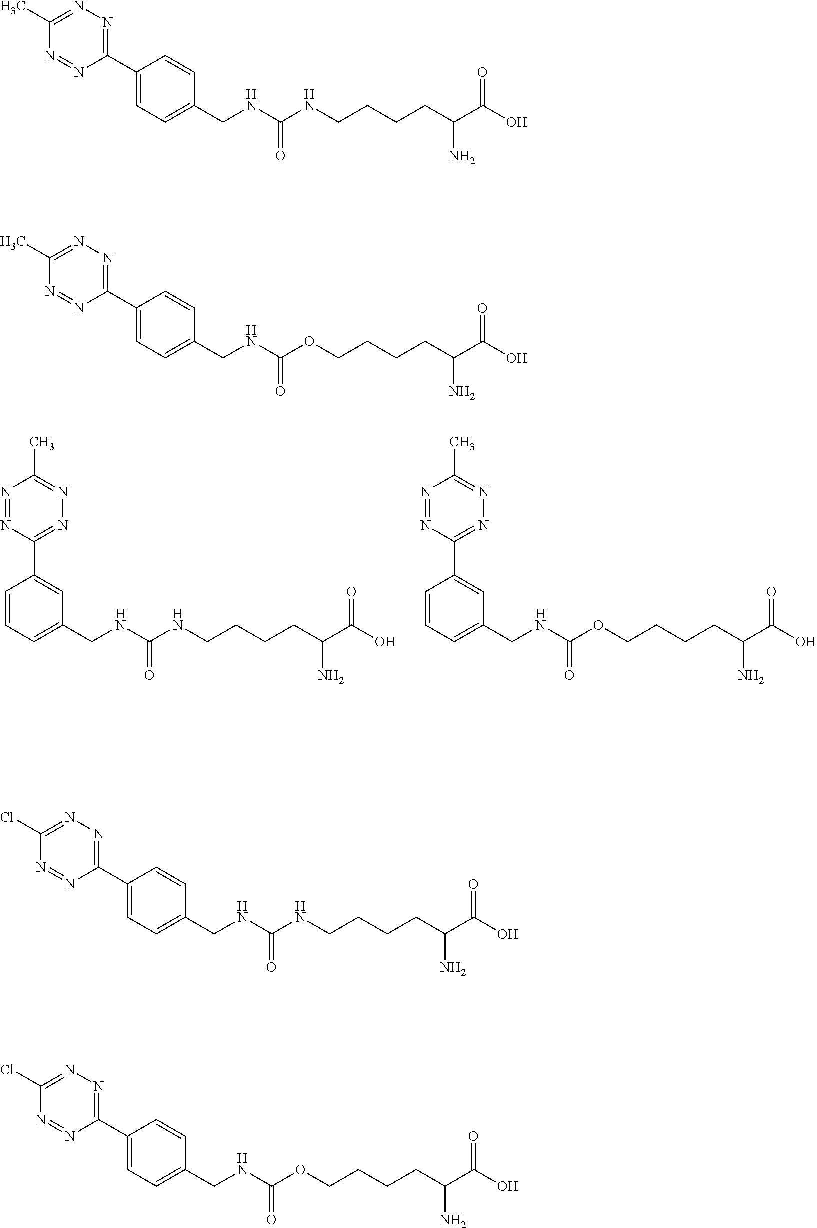 [SMILES: Cc1nnc(-c2ccc(CNC(=O)NCCCCC(N)C(=O)O)cc2)nn1 ; Cc1nnc(-c2ccc(CNC(=O)OCCCCC(N)C(=O)O)cc2)nn1 ; Cc1nnc(-c2cccc(CNC(=O)NCCCCC(N)C(=O)O)c2)nn1 ; Cc1nnc(-c2cccc(CNC(=O)OCCCCC(N)C(=O)O)c2)nn1 ; NC(CCCCNC(=O)NCc1ccc(-c2nnc(Cl)nn2)cc1)C(=O)O ; NC(CCCCOC(=O)NCc1ccc(-c2nnc(Cl)nn2)cc1)C(=O)O]

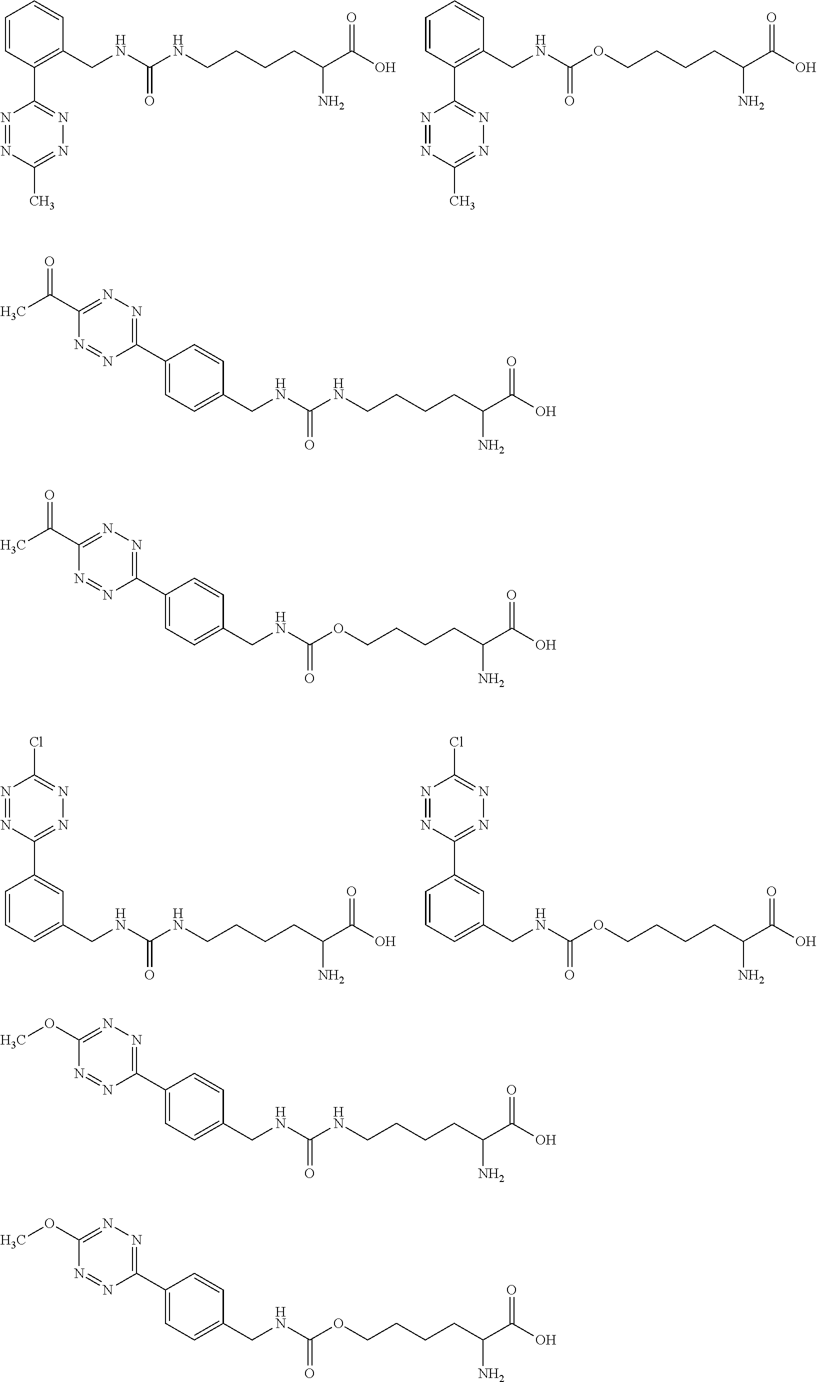

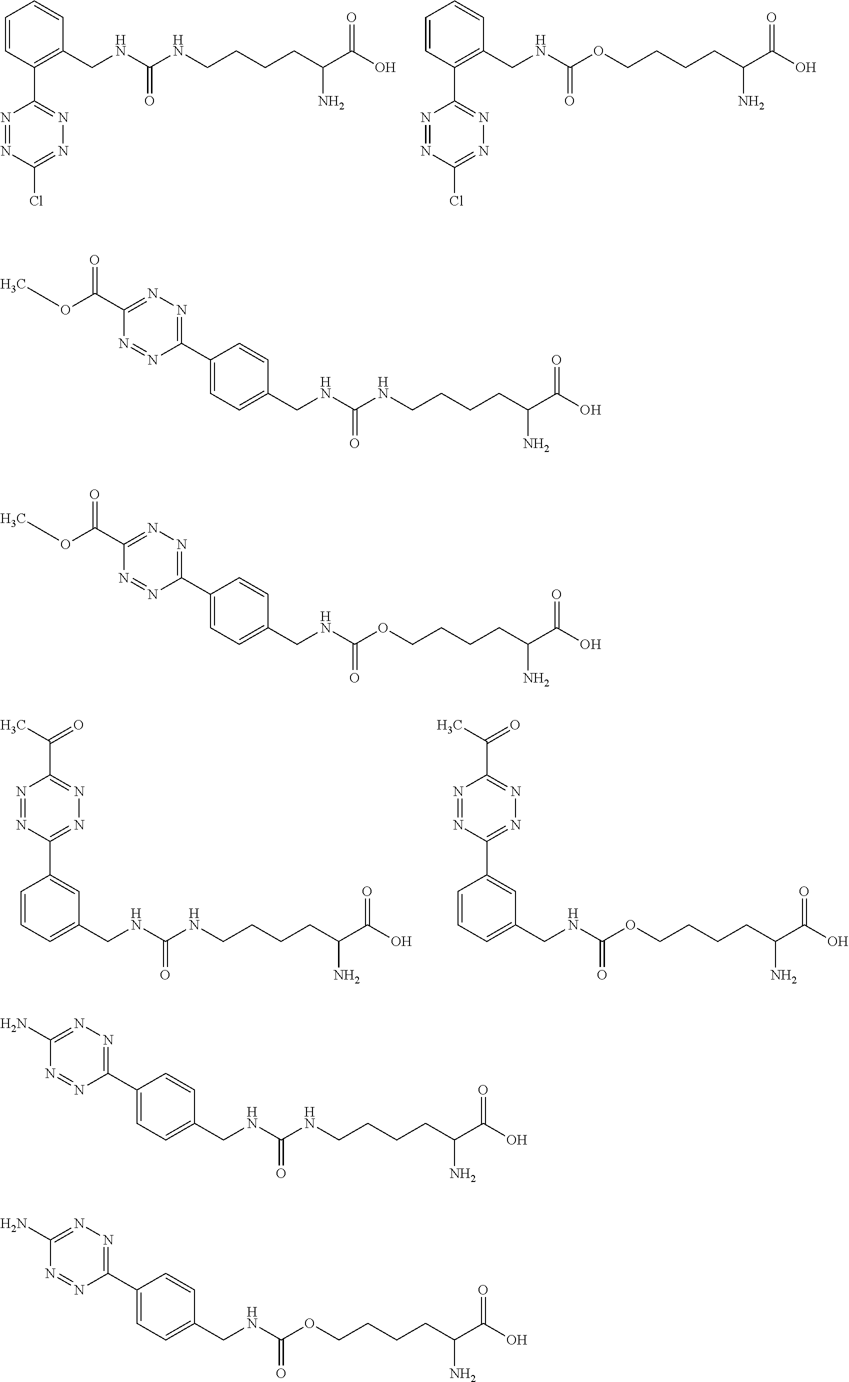

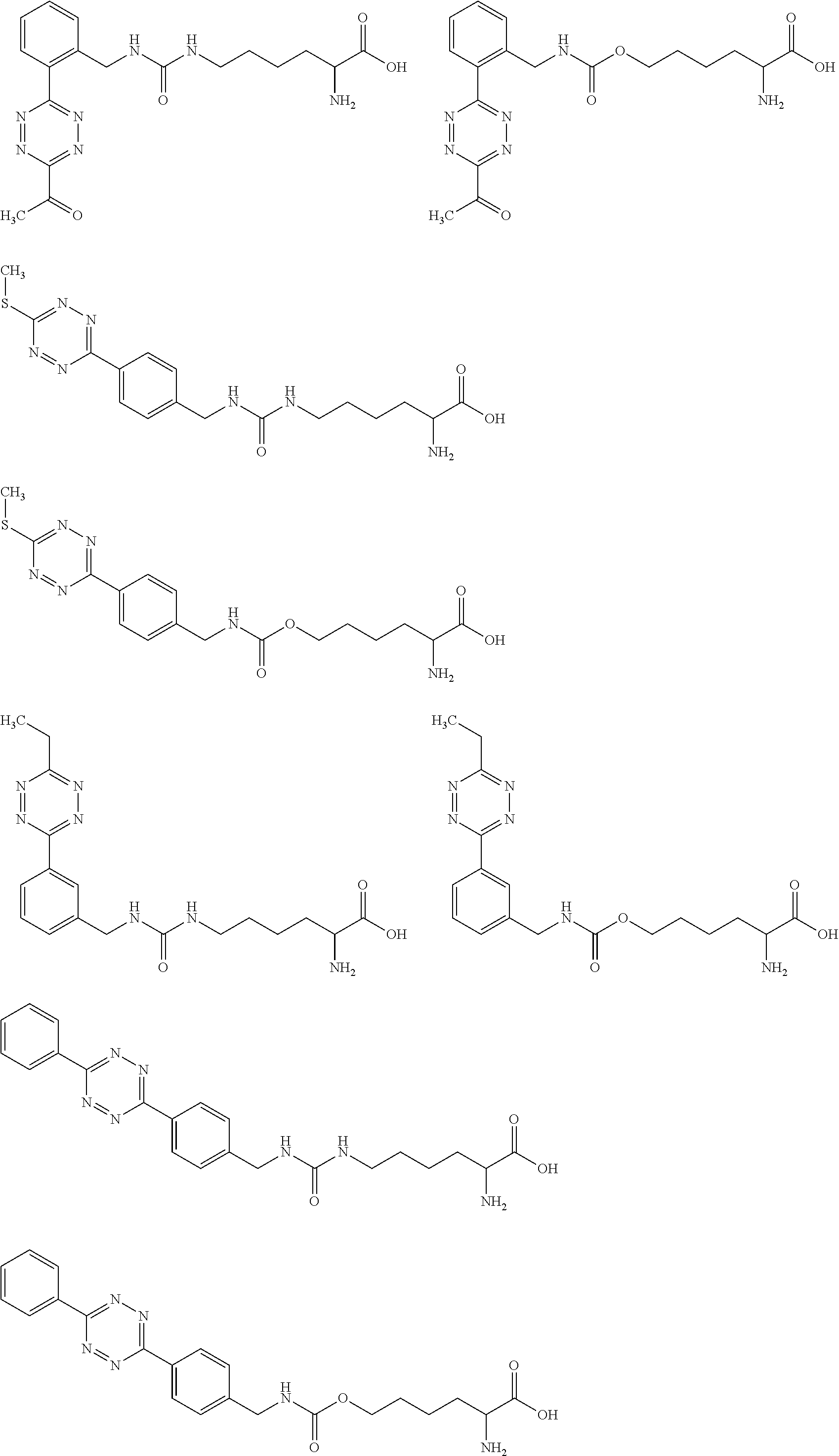

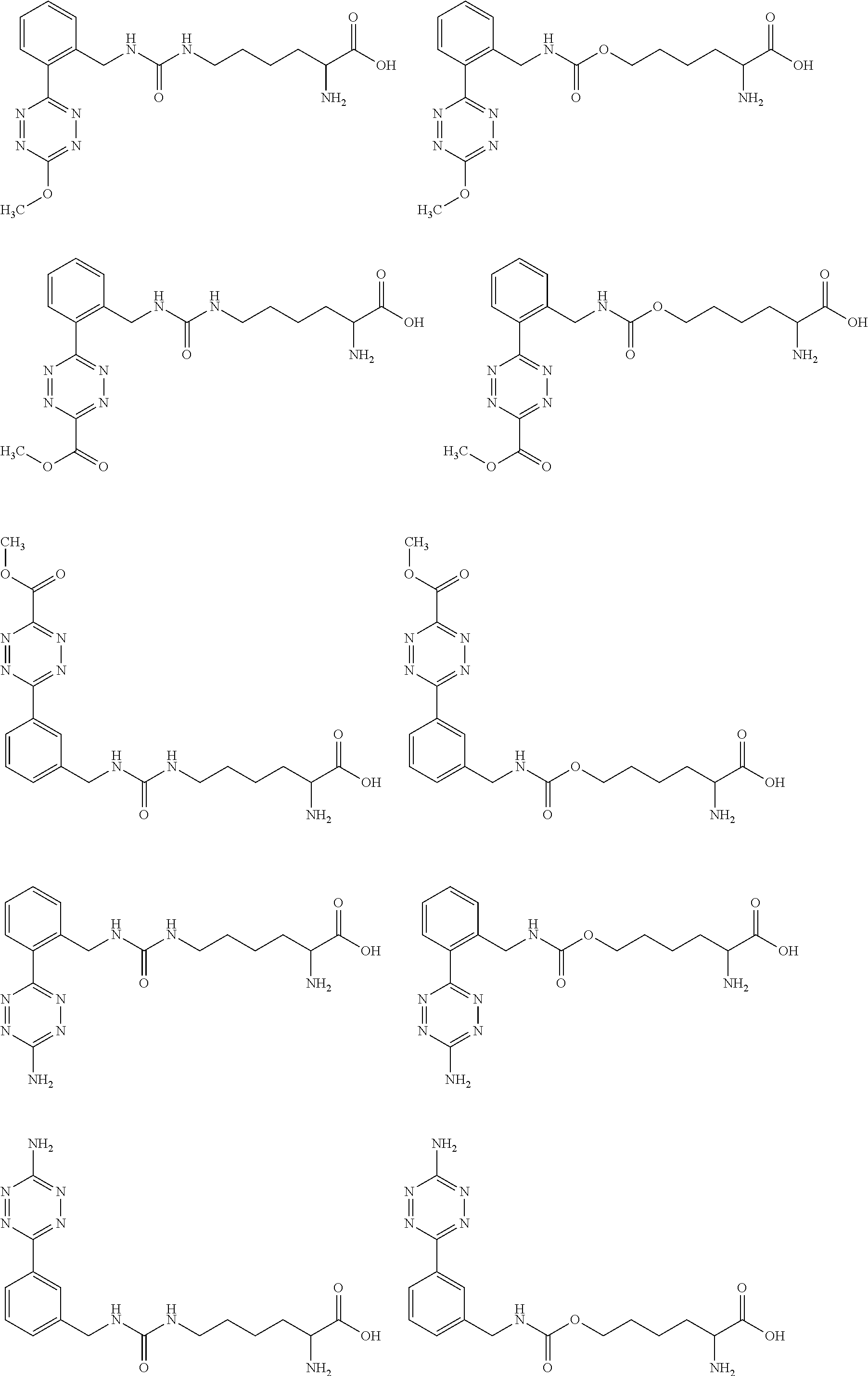

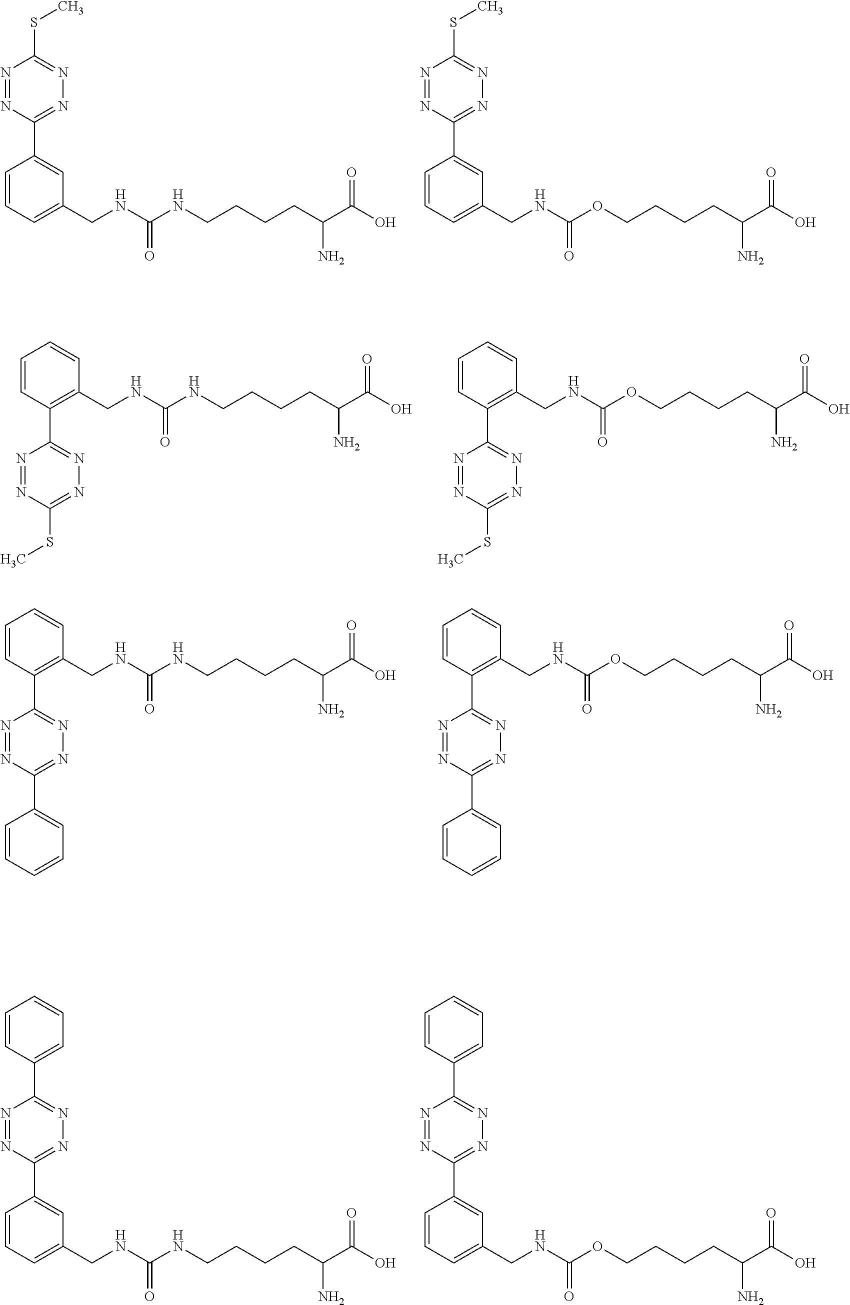

3. The compound according to claim 1, wherein R is methyl.

4. A method for producing compounds of general formula (I), comprising the step of reacting a tetrazine derivative of formula (II) with a lysine derivative of formula (III) 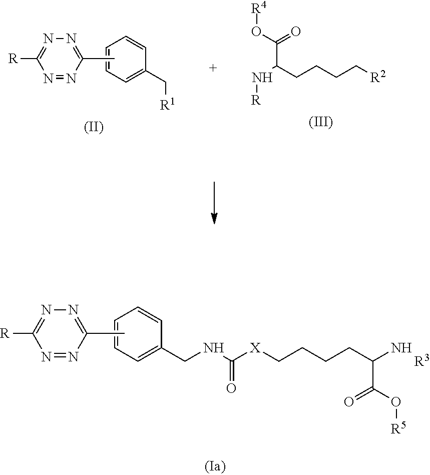
in order to achieve the intermediate compound (Ia), wherein
X denotes N or O,
R is selected from the group consisting of halogen, —OR^{a}, —C(O)R^{a}, —COOR^{a}, —NR^{a}R^{a}, —SR^{a}, —C₁-C₆alkyl and phenyl, wherein the —C₁-C₆alkyl or phenyl moiety is optionally substituted by halogen, —OR^{a}, —C(O)R^{a}, —COOR^{a}, —NR^{a}R^{a}, —SR^{a},
R¹ is —NH₂ or —OCN,
R² is —OH or —NH—C(═O)-imidazol,
R³ is a protecting group,
R⁴ is H or a protecting group and
R⁵ is —OH or —OCH₃.

5. The method of claim 4, wherein the protecting group is selected from the group consisting of tert-butyloxycarbonyl (boc-group), carbobenzyloxy (Cbz) group, p-methoxybenzyl carbonyl (Moz or MeOZ) group, tert-butyloxycarbonyl (BOC) group, 9-fluorenylmethyloxycarbonyl (Fmoc) group, acetyl (Ac), benzoyl (Bz) group, benzyl (Bn) group, carbamate group, p-methoxybenzyl (PMB), 3,4-dimethoxybenzyl (DMPM), p-methoxyphenyl (PMP) group, tosyl (Ts) group, and Troc (trichloroethyl chloroformate).

6. A method for site-specific incorporation of a compound of formula (I) according to any one of claims 1 to 3 into a peptide or protein, comprising the steps of
a. providing a compound of formula (I),
b. providing a cell strain with an orthogonal tRNA-synthetase enzyme with tRNA-charging activity towards the compounds of formula (I),
c. culturing said cell strain in a culture medium comprising the compound of a), and
d. recovering from the culture medium or from the cells obtained in step c) the modified peptide or protein containing at least one compound of formula (I).

7. A modified peptide or protein, wherein the peptide or protein comprises at least one compound of general formula (I).

8. The modified peptide or protein according to claim 7, wherein said at least one compound of formula (I) is incorporated at a desired position of the wild type peptide or protein.

9. The modified peptide or protein according to claim 7 or 8, wherein the tetrazine moiety of said modified peptide or protein is further linked to an electron-rich dienophile compound or a strained dienophile compound.

10. The modified peptide or protein according to claim 9, wherein said electron-rich dienophile compound is selected from the group consisting of a norbornene compound, a cyclopropene compound, a bicyclo[6.1.0]nonyl compound, a trans-cyclooctene compound, a styrene compound or a spirohexene compound.

11. A method of producing a modified peptide or protein, comprising the steps of:
a. providing a modified peptide or protein according to claim 7 or 8 and an electron-rich dienophile compound,
b. incubating said components to allow linkage of the tetrazine moiety of the modified peptide or protein to said electron-rich dienophile compound.

12. The method according to claim 11, wherein said electron-rich dienophile compound is selected from the group comprising a norbornene compound, a cyclopropene compound, and a bicyclo[6.1.0]nonyl compound, a trans-cyclooctene compound, a styrene compound or a spirohexene compound.

13. Use of a compound of general formula (I) for chemical synthesis or as synthon for pharmaceutical ingredients.
